(19) United States
(12) Patent Application Publication
(10) Pub. No.: US 2005/0171023 A1
(43) Pub. Date: Aug. 4, 2005

(54) Caspase inhibitors for the treatment of diseases and conditions caused by exposure to radionuclides, biological agents, or chemical agents

(76) Inventors: Sui Xiong Cai, San Diego, CA (US); Ben Y. Tseng, San Diego, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/510,104

(22) PCT Filed: Apr. 7, 2003
(86) PCT No.: PCT/US03/10645

Related U.S. Application Data
(60) Provisional application No. 60/369,806, filed on Apr. 5, 2002.

(51) Int. Cl. A61K 38/04; A61K 31/198; A61K 31/16

(52) U.S. Cl. 514/19; 514/563; 514/616

(57) ABSTRACT

The use of caspase inhibitors for treating cell death induced by radionuclides, biological agents, or chemical agents is disclosed. In particular, treatment of diseases or conditions caused by exposure to radionuclides, biological agents, or chemical agents, spread of radionuclides, biological agents, or chemical agents, explosion of radionuclides, biological agents, or chemical agents by terrorists or accidental exposure to radionuclides, biological agents, or chemical agents from a nuclear power plant, manufacturing or processing plant, research facility, or hospital is disclosed.

### BACKGROUND OF THE INVENTION

1. Field of the Invention

This invention is in the field of medicinal chemistry. In particular, the invention relates to the use of caspase inhibitors to treat diseases and conditions caused by exposure to radionuclides, biological agents, or chemical agents.

2. Related Art

Organisms eliminate unwanted cells by a process variously known as regulated cell death, programmed cell death or apoptosis. Such cell death occurs as a normal aspect of animal development as well as in tissue homeostasis and aging (Glucksmann, A., Biol. Rev. Cambridge Philos. Soc. 26:59-86 (1951); Glucksmann, A., Archives de Biologie 76:419-437 (1965);

Ellis et al., Dev. 112:591-603 (1991); Vaux et al., Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J. Internal Medicine 237:529-536 (1995)).

Apoptosis is achieved through an endogenous mechanism of cellular suicide (Wyllie, A. H., in Cell Death in Biology and Pathology, Bowen and Lockshin, eds., Chapman and Hall (1981), pp. 9-34). A cell activates its internally encoded suicide program as a result of either internal or external signals. The suicide program is executed through the activation of a carefully regulated genetic program (Wyllie et al., Int. Rev. Cyt. 68: 251 (1980); Ellis et al., Ann. Rev. Cell Bio. 7: 663 (1991)). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 237:529-536 (1995)).

Mammalian interleukin-1β (IL-1β) plays an important role in various pathologic processes, including chronic and acute inflammation and autoimmune diseases (Oppenheim, J. H. et al., Immunology Today, 7, 45-56 (1986)). IL-1β is synthesized as a cell associated precursor polypeptide (pro-IL-1β) that is unable to bind IL-1 receptors and is biologically inactive (Mosley et al., J. Biol. Chem. 262:2941-2944 (1987)). By inhibiting conversion of precursor IL-I1β to mature IL-1β, the activity of interleukin-1 can be inhibited. Interleukin-1β converting enzyme (ICE) is a protease responsible for the activation of interleukin-1β (IL-1β) (Thornberry, N. A., et al., Nature 356: 768 (1992); Yuan, J., et al., Cell 75: 641 (1993)). ICE is a substrate-specific cysteine protease that cleaves the inactive prointerleukin-1 to produce the mature IL-1. The genes that encode for ICE and CPP32 are members of the mammalian ICE/Ced-3 family of genes which presently includes at least twelve members: ICE, CPP32/Yama/Apopain, mICE2, ICE4, ICH1, TX/ICH-2, MCH2, MCH3, MCH4, FLICE/MACH/MCH5, ICE-LAP6 and ICEᵣₑ₁III. The proteolytic activity of this family of cysteine proteases, whose active site (a cysteine residue) is essential for ICE-mediated apoptosis, appears critical in mediating cell death (Miura et al., Cell 75: 653-660 (1993)). This gene family has recently been named caspases (Alnemri, E. S. et al., Cell, 87, 171 (1996), and Thornberry, N. A. et al., J. Biol. Chem. 272, 17907-17911 (1997)) and divided into three groups according to its known functions. Table 1 summarizes these known caspases.

TABLE 1
Enzyme*

Group I: mediators of inflammation
Caspase-1 (ICE)
Caspase-4 (ICEᵣₑₗ-II, TX, ICH-2)
Caspase-5 (ICEᵣₑₗ-III, TY)
Group II: effectors of apoptosis
Caspase-2 (ICH-1, mNEDD2)
Caspase-3 (apopain, CPP-32, YAMA)
Caspase-7 (Mch-3, ICE-LAP3, CMH-1)
Group III: activators of apoptosis
Caspase-6 (Mch2)
Caspase-8 (MACH, FLICE, Mch5)
Caspase-9 (ICE-LAP6, Mch6)
Caspase-10

IL-1 is also a cytokine involved in mediating a wide range of biological responses including inflammation, septic shock, wound healing, hematopoiesis and growth of certain leukemias (Dinarello, C. A., Blood 77:1627-1652 (1991); diGiovine et al., Immunology Today 11:13 (1990)).

WO 93/05071 discloses peptide ICE inhibitors with the formula:
Z-Q₂-Asp-Q₁
wherein Z is an N-terminal protecting group; Q₂ is 0 to 4 amino acids such that the sequence Q₂-Asp corresponds to at least a portion of the sequence Ala-Tyr-Val-His-Asp (SEQ ID NO: 1); Q₁ comprises an electronegative leaving group.

WO 96/03982 discloses aspartic acid analogs as ICE inhibitors with the formula: 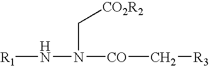
wherein R₂ is H or alkyl; R₃ is a leaving group such as halogen; R₁ is heteroaryl-CO or an amino acid residue.

U.S. Pat. No. 5,585,357 discloses peptidic ketones as ICE inhibitors with the formula: 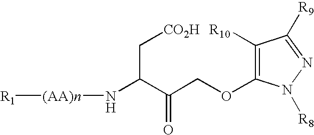
wherein n is 0-2; each AA is independently L-valine or L-alanine; R₁ is selected from the group consisting of N-benzyloxycarbonyl and other groups; R₈, R₉, R₁₀ are each independently hydrogen, lower alkyl and other groups.

Mjalli et al., Bioorg. Med. Chem. Lett. 3:2689-2692 (1993) report the preparation of peptide phenylalkyl ketones as reversible inhibitors of ICE, such as:

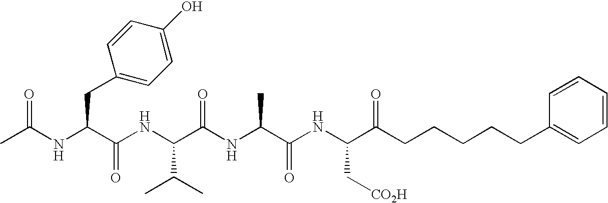

Thornberry et al., Biochemistry 33:3934-3940 (1994) report the irreversible inactivation of ICE by peptide acyloxymethyl ketones: 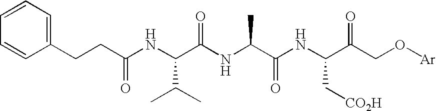
wherein Ar is COPh-2,6-(CF₃)₂, COPh-2,6-(CH₃)₂, Ph-F₅ and other groups.

Dolle et al., J. Med. Chem. 37:563-564 (1994) report the preparation of P₁ aspartate-based peptide α-((2,6-dichlorobenzoyl)oxy)methyl ketones as potent time-dependent inhibitors of ICE, such as:

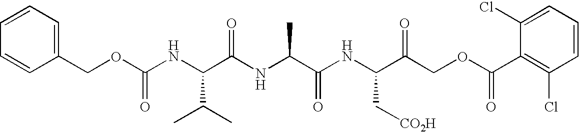

Mjalli et al., Bioorg. Med. Chem. Lett. 4:1965-1968 (1994) report the preparation of activated ketones as potent reversible inhibitors of ICE: 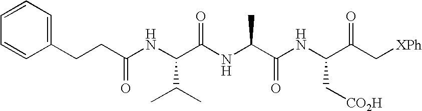
wherein X is NH(CH₂)₂, OCO(CH₂)₂, S(CH₂)₃ and other groups.

Dolle et al., J. Med. Chem. 37:3863-3866 (1994) report the preparation of α-((1-phenyl-3-(trifluoromethyl)-pyrazol-5-yl)oxy)methyl ketones as irreversible inhibitors of ICE, such as:

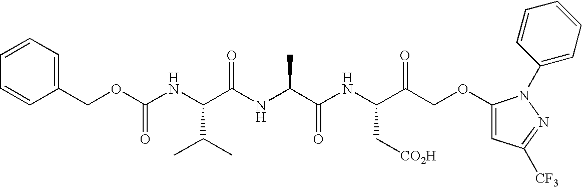

Mjalli et al., Bioorg. Med. Chem. Lett. 5:1405-1408 (1995) report inhibition of ICE by N-acyl-Aspartic acid ketones: 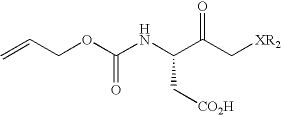
wherein XR₂ is NH(CH₂)₂Ph, OCO(CH₂)₂cyclohexyl and other groups.

Mjalli et al., Bioorg. Med. Chem. Lett. 5:1409-1414 (1995) report inhibition of ICE by N-acyl-aspartyl aryloxymethyl ketones, such as:

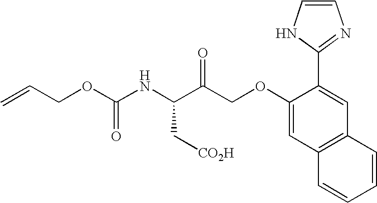

Dolle et al., J. Med. Chem. 38:220-222 (1995) report the preparation of aspartyl α-((diphenylphosphinyl)oxy)methyl ketones as irreversible inhibitors of ICE, such as:

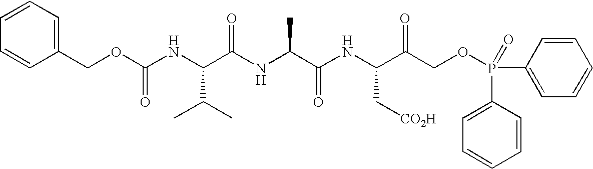

Graybill et al., Bioorg. Med. Chem. Lett. 7:41-46 (1997) report the preparation of α-((tetronoyl)oxy)- and α-((tetramoyl)oxy)methyl ketones as inhibitors of ICE, such as:

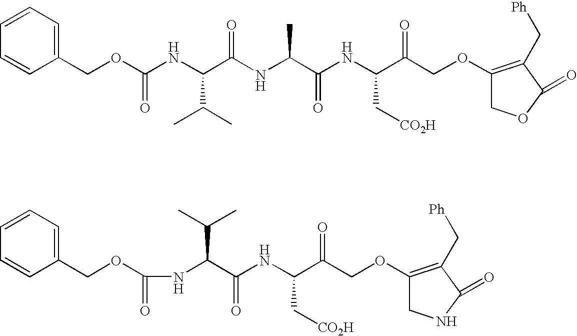

Semple et al., Bioorg. Med. Chem. Lett, 8:959-964 (1998) report the preparation of peptidomimetic aminomethylene ketones as inhibitors of ICE, such as:

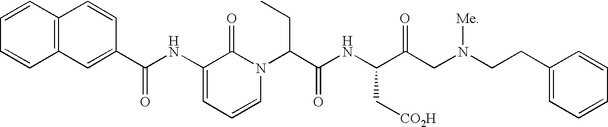

Okamoto et al., Chem. Pharm. Bull. 47:11-21 (1999) report the preparation of peptide based ICE inhibitors with the P1 carboxyl group converted to an amide, such as:

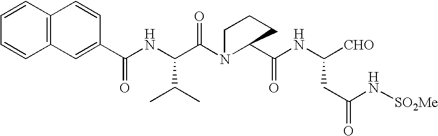

EP 618223 patent application discloses inhibitors of ICE as anti-inflammatory agents:
R-A₁-A₂-X-A₃
Wherein R is a protecting group or optionally substituted benzyloxy; A₁ is an α-hydroxy or α-amino acid residue or a radical of formula: 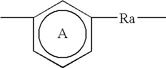
wherein ring A is optionally substituted by hydroxy or C₁₋₄ alkoxy and Rₐ is CO or CS; A₂ is an α-hydroxy or α-amino acid residue or A₁ and A₂ form together a pseudo-dipeptide or a dipeptide mimetic residue; X is a residue derived from Asp; A₃ is —CH₂—X₁—CO—Y₁, —CH₂—O—Y₂, —CH₂—S—Y₃, wherein X₁ is O or S; Y₁, Y₂ or Y₃ is cycloaliphatic residue, and optionally substituted aryl.

WO 99/18781 and U.S. Pat. No. 6,184,210 disclose dipeptides of formula I: 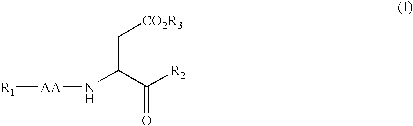
wherein R₁ is an N-terminal protecting group;
AA is a residue of any natural or non-natural α-amino acid, β-amino acid, derivatives of an α-amino acid or β-amino acid;
R₂ is H or CH₂R₄ where R₄ is an electronegative leaving group; and
R₃ is alkyl or H, provided that AA is not His, Tyr, Pro or Phe.
   These dipeptides are surprisingly potent caspase inhibitors of apoptosis in cell based systems. These compounds are systemically active in vivo and are potent inhibitors of antiFas-induced lethality in a mouse liver apoptosis model and have robust neuroprotective effects in a rat model of ischemic stroke.

Exemplary preferred inhibitors of apoptosis include Boc-Ala-Asp-CH₂F, Boc-Val-Asp-CH₂F, Boc-Leu-Asp-CH₂F, Ac-Val-Asp-CH₂F, Ac-Ile-Asp-CH₂F, Ac-Met-Asp-CH₂F, Cbz-Val-Asp-CH₂F, Cbz-β-Ala-Asp-CH₂F, Cbz-Leu-Asp-CH₂F, Cbz-Ile-Asp-CH₂F, Boc-Ala-Asp(OMe)-CH₂F, Boc-Val-Asp(OMe)-CH₂F, Boc-Leu-Asp(OMe)-CH₂F, Ac-Val-Asp(OMe)-CH₂F, Ac-Ile-Asp(OMe)-CH₂F, Ac-Met-Asp(OMe)-CH₂F, Cbz-Val-Asp(OMe)-CH₂F, Cbz-β-Ala-Asp(OMe)-CH₂F, Cbz-Leu-Asp(OMe)-CH₂F and Cbz-Ile-Asp(OMe)-CH₂F; where Boc is tert.-butyoxycarbonyl and Cbz is carbobenzyloxy.

WO 99/47154 and U.S. Pat. No. 6,153,591 disclose dipeptides of formula II: 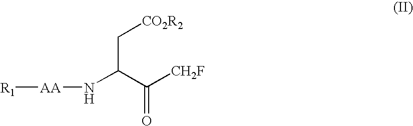
wherein R₁ is an N-terminal protecting group;
AA is a residue of a non-natural α-amino acid or β-amino acid; and
R₂ is an optionally substituted alkyl or H.

Exemplary inhibitors of caspases and apoptosis include Boc-Phg-Asp-fmk, Boc-(2-F-Phg)-Asp-fmk, Boc-(F₃-Val)-Asp-fmk, Boc-(3-F-Val)-Asp-fmk, Ac-Phg-Asp-fmk, Ac-(2-F-Phg)-Asp-fmk, Ac-(F₃-Val)-Asp-fmk, Ac-(3-F-Val)-Asp-fmk, Z-Phg-Asp-fmk, Z-(2-F-Phg)-Asp-fmk, Z-(F₃-Val)-Asp-fmk, Z-Chg-Asp-fmk, Z-(2-Fug)-Asp-fmk, Z-(4-F-Phg)-Asp-fmk, Z-(4-Cl-Phg)-Asp-fmk, Z-(3-Thg)-Asp-fmk, Z-(2-Fua)-Asp-frnk, Z-(2-Tha)-Asp-fmk, Z-(3-Fua)-Asp-fmk, Z-(3-Tha)-Asp-fmk, Z-(3-Cl-Ala)-Asp-fmk, Z-(3-F-Ala)-Asp-fmk, Z-(F₃-Ala)-Asp-fmk, Z-(3-F-3-Me-Ala)-Asp-fmk, Z-(3-Cl-3-F-Ala)-Asp-fmk, Z-(2-Me-Val)-Asp-fmk, Z-(2-Me-Ala)-Asp-fmk, Z-(2-i-Pr-β-Ala)-Asp-fmk, Z-(3-Ph-β-Ala)-Asp-fmk, Z-(3-CN-Ala)-Asp-fmk, Z-(1-Nal)-Asp-fmk, Z-Cha-Asp-fmk, Z-(3-CF₃-Ala)-Asp-fmk, Z-(4-CF₃-Phg)-Asp-fmk, Z-(3-Me₂N-Ala)-Asp-fmk, Z-(2-Abu)-Asp-fmk, Z-Tle-Asp-fmk, Z-Cpg-Asp-fmk, Z-Cbg-Asp-fmk, Z-Thz-Asp-fmk, Z-(3-F-Val)-Asp-fmk, and Z-(2-Thg)-Asp-fmk; where Z is benzyloxycarbonyl, BOC is tert.-butoxycarbonyl, fmk is fluoromethylketone, Ac is acetyl, Phg is phenylglycine, 2-F-Phg is (2-fluorophenyl)glycine, F₃-Val is 4,4,4-trifluoro-valine, 3-F-Val is 3-fluoro-valine, 2-Thg is (2-thienyl)glycine, Chg is cyclohexylglycine, 2-Fug is (2-furyl)glycine, 4-F-Phg is (4-fluorophenyl)glycine, 4-Cl-Phg is (4-chlorophenyl)glycine, 3-Thg is (3-thienyl)glycine, 2-Fua is (2-furyl)alanine, 2-Tha is (2-thienyl)alanine, 3-Fua is (3-furyl)alanine, 3-Tha is (3-thienyl)alanine, 3-Cl-Ala is 3-chloroalanine, 3-F-Ala is 3-fluoroalanine, F₃-Ala is 3,3,3-trifluoroalanine, 3-F-3-Me-Ala is 3-fluoro-3-methylalanine, 3-Cl-3-F-Ala is 3-chloro-3-fluoroalanine, 2-Me-Val is 2-methylvaline, 2-Me-Ala is 2-methylalanine, 2-i-Pr-β-Ala is 3-amino-2-isopropylpropionic acid, 3-Ph-β-Ala is 3-amino-3-phenylpropionic acid, 3-CN-Ala is 3-cyanoalanine, 1-Nal is 3-(1-naphthyl)-alanine, Cha is cyclohexylalanine, 3-CF₃-Ala is 2-amino-4,4,4-trifluorobutyric acid, 4-CF₃-Phg is 4-trifluoromethylphenylglycine, 3-Me₂N-Ala is 3-dimethylamino-alanine, 2-Abu is 2-aminobutyric acid, Tle is tert-leucine, Cpg is cyclopentylglycine, Cbg is cyclobutylglycine, and Thz is thioproline.

WO 00/55114 and U.S. appl. Ser. No. 09/527,225 disclose compounds of formulae III, IV and V: 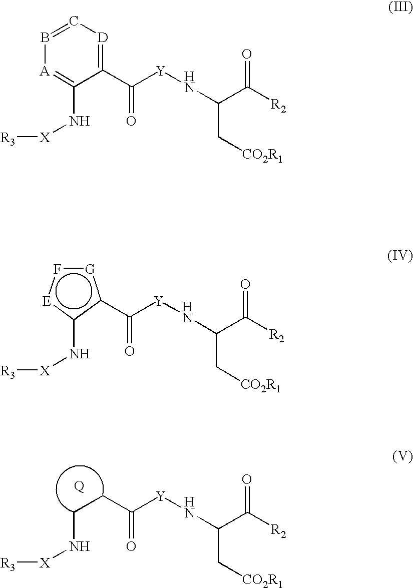
wherein
R₁ is an optionally substituted alkyl or hydrogen;
R₃ as an N-protecting group;
R₂ is hydrogen or optionally substituted alkyl;
A is CR₆ or nitrogen;
B is CR₇ or nitrogen;
C is CR₈ or nitrogen;
D is CR₉ or nitrogen;
provided that not more than two of A, B, C or D is nitrogen; and
R₆-R₉ independently are hydrogen, halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl; C₁-C₆ hydroxyalkyl, nitro, amino, cyano, C₁-C₆ acylamino, hydroxy, C₁-C₆ acyloxy, C₁-C₆ alkoxy, alkylthio, or carboxy; or one of R₆ and R₇, or R₇ and R₈, or R₈ and R₉ are taken together with the carbon atoms to which they are attached to form a carbocycle or heterocycle;
E is CR₁₄, nitrogen, oxygen or sulfur;
F is CR₁₅, nitrogen, oxygen or sulfur;
G is C₁₆, nitrogen, oxygen or sulfur;
provided that only one of E, F, G is nitrogen, oxygen or sulfur, where R₁₄-R₁₆ are independently hydrogen, halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl; C₁-C₆ hydroxyalkyl, nitro, amino, cyano, C₁-C₆ acylamino, hydroxy, C₁-C₆ acyloxy, C₁-C₆ alkoxy, alkylthio, or carboxy; or one of R₁₄ and R₁₅, or R₁₅ and R₁₆, are taken together with the carbon atoms to which they are attached to form a carbocycle or heterocycle;
Q represents an optionally substituted saturated or partially saturated carbocycle or heterocycle;
X is a peptide of 1-4 amino acids or a bond; and
Y is a peptide of 1-4 amino acids or a bond.

Exemplary inhibitors of caspases and apoptosis include 2-(Z-amino)benzoyl-Asp-fmk, 2-(Z-amino)-3-methylbenzoyl-Asp-fmk, 2-(Z-amino)-3,5-dimethylbenzoyl-Asp-fmk, 2-(Z-amino)-4-chlorobenzoyl-Asp-fmk, 2-(Z-amino)-5-chlorobenzoyl-Asp-fmk, 2-(Z-amino)-5-fluorobenzoyl-Asp-fmk, 2-(Z-amino)-6-fluorobenzoyl-Asp-fmk, cis-2-(Z-amino)cyclohexanecarboxyl-Asp-fmk, 2-(Z-amino)-5-methylbenzoyl-Asp-fmk, 2-(Z-amino)-6-methylbenzoyl-Asp-fmk, 2-(Z-amino)-6-chlorobenzoyl-Asp-fmk, 2-(Z-amino)-3-methoxybenzoyl-Asp-fmk, 2-(Z-amino)thiophene-2-carboxyl-Asp-fmk, 2-(methoxycarbonylamino)thiophene-2-carboxyl -Asp-fmk, cis-2-(Z-amino)cyclopentanecarboxyl-Asp-fmk, trans-2-(Z-amino)cyclopentanecarboxyl-Asp-fmk, 2-(Z-amino)benzoyl-Asp-DCB-methylketone, methoxycarbonyl-Val-(2-aminobenzoyl)-Asp-fmk, Z-Glu-(2-aminobenzoyl)-Asp-fmk, and Z-Val-(2-aminobenzoyl)-Asp-fmk.

WO 01/16093 and U.S. Pat. No. 6,495,522 disclose compounds of formula VI: 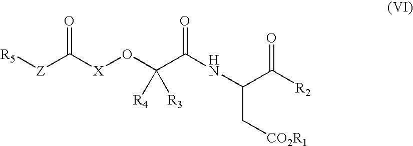
or pharmaceutically acceptable salts or prodrugs thereof, wherein
R₁ is an optionally substituted alkyl or hydrogen;
R₂ is hydrogen or optionally substituted alkyl;
R₃ and R₄ independently are hydrogen, optionally substituted aryl, optionally substituted heterocyclic, optionally substituted carbocyclic, optionally substituted heteroaryl, optionally substituted alkyl, optionally substituted alkenyl, or optionally substituted alkynyl;
R₅ is an optionally substituted alkyl, optionally substituted carbocyclic, optionally substituted heterocyclic, optionally substituted aryl or optionally substituted heteroaryl;
Z is O, S, NR₈, or (CR₉R₁₀)ₙ, where R₈, R₉ and R₁₀ independently are hydrogen, alkyl or cycloalkyl, and n is 0, 1, 2, or 3; and
X is a peptide of 1-2 amino acids or a bond. Where X is one amino acid, it may be any one of the common 20 amino acids e.g., Ala, Val, Leu, Ile, Pro, Phe, Trp, Met, Gly, Ser, Thr, Cys, Tyr, Asp, Asn, Glu, Asn, Lys, Arg and His. Where X is a peptide, it may be Asp-Glu, Asp-Ala, Asp-Phe, Val-Glu, Leu-Glu, Thr-Glu, Ile-Glu, Tyr-Glu, and Trp-Glu.

Exemplary preferred inhibitors of caspases having formula VI include 1-(Carbonyl-Asp-CH₂F)ethyl N-phenylcarbamate, 1-(Carbonyl-Asp-CH₂F)ethyl N-benzylcarbamate, 2-Methyl-1-(carbonyl-Asp-CH₂F)propyl N-phenylcarbamate, 2-Methyl-1-(carbonyl-Asp-CH₂F)propyl N-benzylcarbamate, 2-Methyl-1-(carbonyl-Asp-CH₂F)propyl N-(2,6-dichlorophenyl)carbamate, 2-Methyl-1-(carbonyl-Asp-CH₂F)propyl N-(2,5-dichlorophenyl)-carbamate, 2-Methyl-1-(carbonyl-Asp-CH₂F)propyl N-(2,4-dichlorophenyl)-carbamate, 2-Methyl-1-(carbonyl-Asp-CH₂DCB)propyl N-phenylcarbamate, 2-Methyl-1-(carbonyl-Asp-CH₂DCB)propyl N-(2,6-dichlorophenyl)-carbamate, 2-Methyl-1-(carbonyl-Asp-CH₂PTP)propyl N-phenylcarbamate, 2-Methyl-1-(carbonyl-Asp-CH₂PTP)propyl N-(2,6-dichlorophenyl)-carbamate, 2-Methyl-1-(carbonyl-Asp-CH₂DPP)propyl N-phenylcarbamate, 2-Methyl-1-(carbonyl-Asp-CH₂DPP)propylN-(2,6-dichlorophenyl)-carbamate, 2-Methyl-1-(carbonyl-Asp-CH₂F)propyl N-(2-methyl-1-methoxycarbonyl-propyl)carbamate, 2-Methyl-1-(carbonyl-Asp-CH₂F)propyl N-(3-fluorophenyl)carbamate, 2-Methyl-1-(carbonyl-Asp-CH₂F)propyl N-(4-fluorophenyl)carbamate, 2-Methyl-1-(carbonyl-Asp-CH₂F)propyl N-(3,4-difluorophenyl)carbamate, 2-Methyl-1-(carbonyl-Asp-CH₂F)propyl N-(4-phenoxyphenyl)carbamate, 1-(Carbonyl-Asp-CH₂F)propyl N-phenylcarbamate, 1-(Carbonyl-Asp-CH₂F)butyl N-phenylcarbamate, 1-(Carbonyl-Asp-CH₂F)-2-propenyl N-phenylcarbamate, 2-(4-Imidazolyl)-1-(carbonyl-Asp-CH₂F)ethyl N-phenylcarbamate, 2-Phenyl-1-(carbonyl-Asp-CH₂F)ethyl N-phenylcarbamate, 2-Methyl-1-(carbonyl-Asp-CH₂F)butyl N-phenylcarbamate, 3-Methyl-1-(carbonyl-Asp-CH₂F)butyl N-phenylcarbamate, 1-Phenyl-1-(carbonyl-Asp-CH₂F)methyl N-phenylcarbamate, 1-(2-Chlorophenyl)-1-(carbonyl-Asp-C₂F)methyl N-phenylcarbamate, 1-(4-Chlorophenyl)-1-(carbonyl-Asp-CH₂F)m ethyl N-phenylcarbamate, 1-Cyclohexyl-1-(carbonyl-Asp-CH₂F)methyl N-phenylcarbamate, 2-Chloro-1-(carbonyl-Asp-CH₂F)ethyl N-phenylcarbamate, 2,2,2-Trifluoro-1-(carbonyl-Asp-CH₂F)ethyl N-phenylcarbamate, and Z-Valine 2-methyl-1-(carbonyl-Asp-CH₂F)propyl ester; where DCB is 2,6-dichlorobenzoyloxy, PTP is 1-phenyl-3-(trifluoromethyl)pyrazol-5-yloxy, and DPP is diphenylphosphinyloxy.

WO 00/61542 and U.S. Pat. No. 6,355,618 disclose compounds of formula VII: 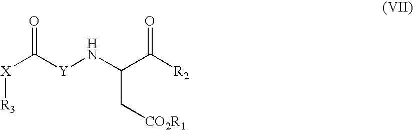
or pharmaceutically acceptable salts or prodrugs thereof, wherein:
R₁ is an optionally substituted alkyl or hydrogen;
R₂ is hydrogen or optionally substituted alkyl;
R₃ is an alkyl, saturated carbocyclic, partially saturated carbocyclic, aryl, saturated heterocyclic, partially saturated heterocyclic or heteroaryl group, wherein said group is optionally substituted;
X is O, S, NR₄, or (CR₄R₅)ₙ, where R₄ and R₅ are, at each occurrence, independently selected from the group consisting of hydrogen, alkyl and cycloalkyl, and n is 0, 1, 2, or 3; or X is NR₄, and R₃ and R₄ are taken together with the nitrogen atom to which they are attached to form a saturated heterocyclic, partially saturated heterocyclic or heteroaryl group, wherein said group is optionally substituted; or
X is CR₄R₅, and R₃ and R₄ are taken together with the carbon atom to which they are attached to form a saturated carbocyclic, partially saturated carbocyclic, aryl, saturated heterocyclic, partially saturated heterocyclic or oxygen-containing heteroaryl group, wherein said group is optionally substituted; and
Y is a residue of a natural or non-natural amino acid;
provided that when X is O, then R₃ is not unsubstituted benzyl or t-butyl; and when X is CH₂, then R₃ is not hydrogen.

Exemplary preferred inhibitors of caspases having formula VII include 2-Chlorobenzyloxycarbonyl-Val-Asp-fmk, 3-Chlorobenzyloxycarbonyl-Val-Asp-fmk, 4-Chlorobenzyloxycarbonyl-Val-Asp-fmk, Phenethoxycarbonyl-Val-Asp-fmk, Cyclohexylmethoxycarbonyl-Val-Asp-fmk, Methoxycarbonyl-Val-Asp-fmk, Ethoxycarbonyl-Val-Asp-fmk, Isopropyloxycarbonyl-Val-Asp-fmk, 2-Chlorobenzyloxycarbonyl-Ile-Asp-fmk, 3-Chlorobenzyloxycarbonyl-Ile-Asp-fmk, 4-Chlorobenzyloxycarbonyl-Ile-Asp-fmk, Phenylacetyl-Val-Asp-fmk, 4-Nitrobenzyloxycarbonyl-Val-Asp-fmk, 2,5-Dimethylbenzyloxycarbonyl-Val-Asp-fmk, 3,4-Dichlorobenzyloxycarbonyl-Val-Asp-fmk, 3,5-Dichlorobenzyloxycarbonyl-Val-Asp-fmk, 2,5-Dichlorobenzyloxycarbonyl-Val-Asp-fmk, 2,6-Dichlorobenzyloxycarbonyl-Val-Asp-fmk, 2,4-Dichlorobenzyloxycarbonyl-Val-Asp-fmk, 2,4-Dimethylbenzyloxycarbonyl-Val-Asp-fmk, 4-Ethylbenzyloxycarbonyl-Val-Asp-fmk, 4-Bromobenzyloxycarbonyl-Val-Asp-fmk, 4-Fluorobenzyloxycarbonyl-Val-Asp-fmk, Cyclopentylmethoxycarbonyl-Val-Asp-fmk, 4-Trifluoromethylbenzyloxycarbonyl-Val-Asp-fmk, 3-Phenylpropionyl-Val-Asp-fmk, Benzylaminocarbonyl-Val-Asp-fmk, 3-Phenylpropyloxycarbonyl-Val-Asp-fmk, 2,4-Difluorobenzyloxycarbonyl-Val-Asp-fmk, 3,4-Difluorobenzyloxycarbonyl-Val-Asp-fmk, 4-Morpholinecarbonyl-Val-Asp-fmk, 4-Pyridylmethoxycarbonyl-Val-Asp-fmk, 2-Pyridylmethoxycarbonyl-Val-Asp-fmk, 2,6-Dichlorobenzyloxycarbonyl-Val-Asp-DCB-methylketone, Isobutoxycarbonyl-Val-Asp-fmk, Propionyl-Val-Asp-fmk, Benzyl-glutaryl-Val-Asp-fmk,Glutaryl-Val-Asp-fmk, 3-(2-Phenyloxyphenyl)propionyl-Val-Asp-fmk, 3-(5-Bromo-2-hydroxyphenyl)propionyl-Val-Asp-fmk, 3-Fluorobenzyloxycarbonyl-Val-Asp-fmk, 2-Fluorobenzyloxycarbonyl-Val-Asp-fmk, 3-Methylbenzyloxycarbonyl-Val-Asp-fmk, 2-Chloro-4-fluorobenzyloxycarbonyl-Val-Asp-fmk, 2-Naphthylmethoxycarbonyl-Val-Asp-fmk, p-Toluenesulfonyl-Val-Asp-fmk, and p-Toluenesulfonyl-Phe-Asp-fmk, where fmk is fluoromethylketone and DCB is 2,6-dichlorobenzoyloxy.

### SUMMARY OF THE INVENTION

Radiation is known to cause apoptotic cell death (Paris et al., Science 293:293-7 (2001) and Sheikh et al., Oncogene 17:2555-2563 (1998)). The present invention arises out of the discovery that caspase inhibitors, which can inhibit apoptosis, are useful for the treatment of radionuclide-induced cell death. Therefore, this invention is useful for the treatment of diseases and conditions, including death, caused by exposure to radionuclides, spread of radionuclides, so called “dirty bombs” exploded by terrorists, or accidental exposure to radionuclides from nuclear power plants, nuclear research facilities or hospitals. Furthermore, this invention is useful for the protection of cells surrounding a treatment site during treatment of cancer or other conditions with radiopharmaceutical agents and the protection of cells during administration of radiolabeled imaging agents. These types of radionuclide exposure are distinguished from therapeutic radiation treatment, as exemplified by radiation therapy for cancer.

Many biological agents, such as those pathogens and toxins that have been used to make biological weapons, including anthrax (Park et al., Science 297:2048-2051 (2002) and Popov et al., FEBS Lett. 527:211-215 (2002)), botulinum (Rohrbach et al., Ann. Otol. Rhinol. Laryngol. 110:1045-1050 (2001) and Doggweiler et al., Prostate 37:44-50 (1998)), aflatoxin (Sun et al., Biomed. Environ. Sci. 15:145-152 (2002) and Meki et al., Neuroendocrinol. Lett. 22:417-426 (2001)), Clostridium (Brito et al., J. Infect. Dis. 186:1438-1447 (2002) and Qa'Dan et al., Cell. Microbiol. 4:425-434 (2002)), plague (Yersinia pestis) (Weeks et al., Microb. Pathol. 32:227-237 (2002), Cornelis, Proc. Natl. Acad. Sci. USA 97:8778-8783 (2000) and Mills et al., Proc. Natl. Acad. Sci. USA 94:12638-12643 (1997)), hemorrhagic fevers (Ebola and Marburg) (Baize et al., Apoptosis 5:5-7 (2000), Geisbert et al., Lab. Invest. 80:171-186 (2000) and Baize et al., Nature Med. 5:423-426 (1999)), Staphylococcus (Mempel et al., Br. J. Dermatol. 146:943-951 (2002) and Kerro et al., Vet. Q. 24:181-198 (2002)), Streptococcus (Kemp et al., Infect. Immun. 70:5019-5025 (2002) and Buratta et al., FEBS Lett. 520:68-72 (2002)), ricin, modeccin, diphtheria, and Pseudomonas (Gan et al., Acta Pharmacol. Sin. 21:243-248 (2000), Hasegawa et al., Biosci. Biotechnol. Biochem. 64:1422-1429 (2000) and Komatuu, J. Biochem. (Tokyo) 124:1038-1044 (1998)), and cholera (Pitman et al., Biochem. Soc. Trans. 26:S338 (1998) and Allam et al., Cancer Res. 57:2615-2618 (1997)), are known to induce apoptosis in cells. Therefore, caspase inhibitors, which can inhibit apoptosis, are useful for the treatment of cell death induced by biological agents, including those mentioned herein above. This invention is useful for the treatment of diseases and conditions, including death, caused by exposure to biological agents, including spread of biological agents by terrorists or accidental exposure to biological agents from manufacturing or processing plants, research facilities, or hospitals.

Many chemical agents, such as those that have been used to make chemical weapons, including nitrogen mustard (Cai et al., Mol. Cancer Ther. 1:21-28 (2001), Ardelt et al., Int. J. Oncol. 18:849-853 (2001) and Pette et al., Immunopharmacology 30:59-69 (1995), and cyanide (Li et al., Toxicol. Appl. Pharmacol. 185:55-63 (200) and Prabhakaran et al., J. Pharmacol. Exp. Ther. 303:510-519 (2002)), are known to induce apoptosis in cells. Therefore, caspase inhibitors, which can inhibit apoptosis, are useful for the treatment of cell induced by chemical agents, including those mentioned herein above. This invention is useful for the treatment of diseases and conditions, including death, caused by exposure to chemical agents, including spread of chemical agents by terrorists or accidental exposure to chemical agents from manufacturing or processing plants, research facilities, or hospitals.

Specifically, compounds useful in the present invention are small molecule caspase inhibitors. These inhibitors include, but are not limited to those described herein and, in particular, those described in U.S. Pat. Nos. 6,153,591, 6,184,210, 6,355,618 and 6,495,522; and international patent application number WO 00/55114.

### DETAILED DESCRIPTION OF THE INVENTION

The invention relates to a method of treating diseases and conditions resulting from exposure to radionuclides, biological agents, or chemical agents comprising administering to the animal in need thereof an effective amount of a caspase inhibitor.

When animals are exposed to radionuclides, one result is the apoptotic death of rapidly dividing cells. Such cells include cells of the gastrointestinal tract, skin, hair, and bone marrow cells. According to the present invention, caspase inhibitors are administered to such cells to prevent apoptosis of such cells. In a preferred embodiment, the caspase inhibitors are administered locally, e.g. to the gastrointestinal tract, mouth, skin or scalp to prevent apoptosis of the gastrointestinal, mouth, skin or hair cells. In another preferred embodiment the caspase inhibitors are administered systemically, e.g., intravenously, intraperitoneally, intramuscularly, or subcutaneously.

Exposure to radionuclides can occur unintentionally, such as by accidental exposure at a facility where radioactive materials are handled, including nuclear power plants, nuclear research facilities and hospitals. Exposure to radionuclides can also be intentional, for example due to explosion of a “dirty” bomb by terrorists or during the process of cleanup of a radioactive spill. Radionuclides can be administered to an animal in the form of radiopharmaceutical agents or radiolabeled imaging agents. These types of exposure are distinguished from exposure of patients to measured doses of radiation for therapeutic reasons, such as radiation treatment of cancer.

Radionuclide exposure includes localized exposure and whole body exposure. Such radionuclides include, but not limited to, actinium (²²⁵Ac), americium (²⁴¹Am), antimony (¹²⁴Sb, ¹²⁵Sb), arsenic (⁷²As, ⁷³As, ⁷⁴As), astatine (²¹¹At), barium (¹⁰³Ba, ¹⁴⁰Ba), beryllium (⁷Be), bismuth (²⁰⁶Bi, ²⁰⁷Bi, ²¹²Bi, ²¹³Bi), bromine (⁷⁷Br), cadmium (¹⁰⁹Cd, ¹¹⁵Cd), calcium (⁴⁵Ca), carbon (¹⁴C), cerium (¹³⁹Ce, ¹⁴¹Ce, ¹⁴⁴Ce), cesium (¹²⁹Cs, ¹³⁷Cs), chromium (⁵¹Cr, ⁵⁶Cr), cobalt (⁵⁵Co, ⁵⁶Co, ⁵⁷Co, ⁵⁸Co, ⁶⁰Co, ⁶⁴Co), copper (⁶¹Cu, ⁶⁴Cu, ⁶⁷Cu), erbium (¹⁶⁹Er), europium (¹⁵²Eu), fluorine (¹⁸F), gadolinium (¹⁵³Gd), gallium (⁶⁷Ga, ⁶⁸Ga), gold (¹⁹⁵Au, ¹⁹⁸Au, ¹⁹⁹Au), hafnium (¹⁷⁵Hf, ¹⁸¹Hf), holmium (¹⁶⁶Ho), hydrogen (³H), krypton (⁸⁵Kr), iodine(¹²³I, ¹²⁵I, ¹²⁶I, ¹³¹I, ¹³³I), indium (¹¹¹In, ¹¹³In), iridium (¹⁹²Ir), iron (⁵²Fe, ⁵⁵Fe, ⁵⁹Fe), lead (²⁰³Pb, ²¹⁰Pb, ²¹²Pb), lutetium (¹⁷⁷Lu), magnesium (⁵²Mg), manganese (⁵⁴Mn), mercury (¹⁹⁷Hg, ²⁰³Hg), molybdenum (⁹⁹Mo), neodynium (¹⁴⁷Nd), neptunium (²³⁷Np), nickel (⁵⁷Ni, ⁶³Ni), niobium (⁹⁵Nb), osmium (¹⁸⁵Os, ¹⁹¹Os), palladium (¹⁰³Pd, ¹⁰⁹Pd), phosphorus (³²P, ³³P), platinum (¹⁹⁵Pt, ¹⁹⁷Pt), plutonium (²³⁹Pu), potassium (⁴⁰K), praseodynium (¹⁴²Pr, ¹⁴³Pr), promethium (¹⁴⁷Pm), protactinium (²³³Pa), radium (²²³Ra, ²²⁶Ra), rhenium (¹⁸⁶Re, ¹⁸⁸Re), rhodium (¹⁰⁵Rh), rubidium (⁸¹Rb, ⁸⁶Rb), ruthenium (⁹⁵Ru, ⁹⁷Ru, ¹⁰³Ru, ¹⁰⁵Ru, ¹⁶Ru), samarium (¹⁵³Sm), scandium (⁴⁴Sc, ⁴⁶Sc, ⁴⁷Sc), selenium (⁷²Se, ⁷³Se, ⁷⁵Se), silver (¹⁰⁰Ag, ¹¹¹Ag), sodium (²²Na), strontium (⁸⁵Sr, ⁸⁹Sr, ⁹⁰Sr), sulfur (³⁵S), tantalum (¹⁷⁹Ta, ¹⁸²Ta), technetium (⁹⁹Tc), tellurium (¹²¹Te, ¹²²Te, ¹²⁵Te, ¹³²Te), terbium (¹⁶¹Tb), thalium (¹⁷⁰Tl, ²⁰¹Tl, ²⁰⁴Tl), thorium (²²⁸Th, ²³⁰Th, ²³²Th), thulium (¹⁶⁵Tm, ¹⁶⁷Tm, ¹⁶⁸Tm, ¹⁷⁰Tm), tin (¹¹³Sn), titanium (⁴⁴Ti), tungsten (¹⁸⁵W), uranium(²³³U, ²³⁵U, ²³⁸U), vanadium (⁴⁸V, ⁴⁹V), ytterbium (¹⁶⁹Yb), yttrium (⁸⁸Y, ⁹⁰Y, ⁹¹Y), zinc (⁶²Zn, ⁶⁵Zn) and zirconium (⁹⁵Zr).

Cells shown to be sensitive to exposure to biological agents or chemical agents include immune system cells (e.g., lymphocytes, macrophages), skin cells, endothelial cells, mucosal cells, liver cells and neuronal cells. According to the present invention, caspase inhibitors are administered to such cells to prevent apoptosis of such cells. In a preferred embodiment, the caspase inhibitors are administered locally, e.g. to the gastrointestinal tract, mouth, skin or scalp to prevent apoptosis of the gastrointestinal, mouth, skin or hair cells. In another preferred embodiment the caspase inhibitors are administered systemically, e.g., intravenously, intraperitoneally, intramuscularly, or subcutaneously

Exposure to biological agents or chemical agents can occur unintentionally, such as by accidental exposure at a facility where biological agents to chemical agents are handled, including manufacturing or processing plants, research facilities or hospitals. Exposure to biological agents or chemical agents can also be intentional, for example due to explosion or spread of biological or chemical weapons by terrorists or during the process of cleanup of a biological or chemical spill.

Exposure to biological agents includes localized exposure and whole body exposure. Biological agents (pathogens and toxins) include, but are not limited to, anthrax and its toxins, botulinum and its toxins, aflatoxin (such as aflatoxin G1, aflatoxin B1), sterigmatocystin, deoxynivalenol, fumonisin B1, Clostridium dificile and its toxins, plague (Yersinia pestis) and its toxins, hemorrhagic fevers (Ebola and Marburg), Staphylococcus aureus, Streptococcus (Group A and Group B, GAS and GBS), ricin, modeccin, diphtheria, Pseudomonas, and cholera and its toxins.

Exposure to chemical agents includes localized exposure and whole body exposure. Chemical agents include, but are not limited to, phosphoramide mustard, melphalan, chlorambucil, quinacrine mustard, nitrogen mustard, cyclophosphamide, 4-hydroxycyclophosphamide, and cyanide.

In a preferred embodiment, the caspase inhibitor has the formula: 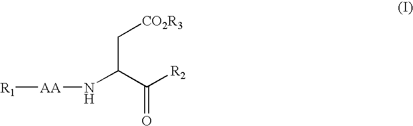
or a pharmaceutically acceptable salt thereof;
wherein R₁ is an N-terminal protecting group;
AA is a residue of any natural or non-natural α-amino acid, β-amino acid, derivatives of an α-amino acid or β-amino acid;
R₂ is H or CH₂R₄ where R₄ is an electronegative leaving group; and
R₃ is alkyl or H.

Examples of such caspase inhibitors include Boc-Ala-Asp-CH₂F, Boc-Val-Asp-CH₂F, Boc-Leu-Asp-CH₂F, Ac-Val-Asp-CH₂F, Ac-Ile-Asp-CH₂F, Ac-Met-Asp-CH₂F, Cbz-Val-Asp-CH₂F, Cbz-P-Ala-Asp-CH₂F, Cbz-Leu-Asp-CH₂F, Cbz-Ile-Asp-CH₂F, Boc-Ala-Asp(OMe)-CH₂F, Boc-Val-Asp(OMe)-CH₂F, Boc-Leu-Asp(OMe)-CH₂F, Ac-Val-Asp(OMe)-CH₂F, Ac-Ile-Asp(OMe)-CH₂F, Ac-Met-Asp(OMe)-CH₂F, Cbz-Val-Asp(OMe)-CH₂F, Cbz-β-Ala-Asp(OMe)-CH₂F, Cbz-Leu-Asp(OMe)-CH₂F or Cbz-Ile-Asp(OMe)-CH₂F.

In another preferred embodiment, the caspase inhibitor has the formula II: 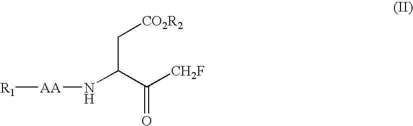
or a pharmaceutically acceptable salt thereof,
wherein R₁ is an N-terminal protecting group;
AA is a residue of a non-natural α-amino acid or β-amino acid; and
R₂ is an optionally substituted alkyl or H.

Examples of such caspase inhibitors include Boc-Phg-Asp-fmk, Boc-(2-F-Phg)-Asp-fmk, Boc-(F₃-Val)-Asp-fmk, Boc-(3-F-Val)-Asp-fmk, Ac-Phg-Asp-fmk, Ac-(2-F-Phg)-Asp-fmk, Ac-(F₃-Val)-Asp-fmk, Ac-(3-F-Val)-Asp-fmk, Z-Phg-Asp-fmk, Z-(2-F-Phg)-Asp-fmk, Z-(F₃-Val)-Asp-fmk, Z-Chg-Asp-fmk, Z-(2-Fug)-Asp-fmk, Z-(4-F-Phg)-Asp-fmk, Z-(4-Cl-Phg)-Asp-fmk, Z-(3-Thg)-Asp-fmk, Z-(2-Fua)-Asp-fmk, Z-(2-Tha)-Asp-fmk, Z-(3-Fua)-Asp-fmk, Z-(3-Tha)-Asp-fmk, Z-(3-Cl-Ala)-Asp-fmk, Z-(3-F-Ala)-Asp-fmk, Z-(F₃-Ala)-Asp-fmk, Z-(3-F-3-Me-Ala)-Asp-fmk, Z-(3-Cl-3-F-Ala)-Asp-fmk, Z-(2-Me-Val)-Asp-fmk, Z-(2-Me-Ala)-Asp-fmk, Z-(2-i-Pr-β-Ala)-Asp-fmk, Z-(3- -Ph-β-Ala)-Asp-fmk, Z-(3-CN-Ala)-Asp-fmk, Z-(1-Nal)-Asp-fmk, Z-Cha-Asp-fmk, Z-(3-CF₃-Ala)-Asp-fmk, Z-(4-CF₃-Phg)-Asp-fmk, Z-(3-Me₂N-Ala)-Asp-fmk, Z-(2-Abu)-Asp-fmk, Z-Tle-Asp-fmk, Z-Cpg-Asp-fmk, Z-Cbg-Asp-fmk, Z-Thz-Asp-fmk, Z-(3-F-Val)-Asp-fmk, and Z-(2-Thg)-Asp-fmk.

In another preferred embodiment, the caspase inhibitor has the formula of one of formulae III, IV and V: 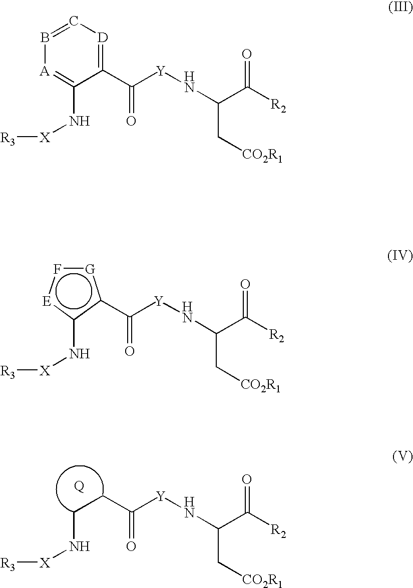
or a pharmaceutically acceptable salt thereof;
wherein R₁ is an optionally substituted alkyl or hydrogen;
R₃ is an N-protecting group;
R₂ is hydrogen or optionally substituted alkyl;
A is CR₆ or nitrogen;
B is CR₇ or nitrogen;
C is CR₈ or nitrogen;
D is CR₉ or nitrogen;
provided that not more than two of A, B, C or D is nitrogen; and
R₆-R₉ independently are hydrogen, halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl; C₁-C₆ hydroxyalkyl, nitro, amino, cyano, C₁-C₆ acylamino, hydroxy, C₁-C₆ acyloxy, C₁-C₆ alkoxy, alkylthio, or carboxy; or
one of R₆ and R₇, or R₇ and R₈, or R₈ and R₉ are taken together with the carbon atoms to which they are attached to form a carbocycle or heterocycle;
E is CR₁₄, nitrogen, oxygen or sulfur;
F is CR₁₅, nitrogen, oxygen or sulfur;
G is C₁₆, nitrogen, oxygen or sulfur;
provided that only one of E, F, G is nitrogen, oxygen or sulfur, where R₁₄-R₁₆ are independently hydrogen, halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl; C₁-C₆ hydroxyalkyl, nitro, amino, cyano, C₁-C₆ acylamino, hydroxy, C₁-C₆ acyloxy, C₁-C₆ alkoxy, alkylthio, or carboxy; or
one of R₁₄ and R₁₅, or R₁₅ and R₁₆, are taken together with the carbon atoms to which they are attached to form a carbocycle or heterocycle;
Q represents an optionally substituted saturated or partially saturated carbocycle or heterocycle;
X is a peptide of 1-4 amino acids or a bond; and
Y is a peptide of 1-4 amino acids or a bond.

Examples of such caspase inhibitors include 2-(Z-amino)benzoyl-Asp-fmk, 2-(Z-amino)-3-methylbenzoyl-Asp-fmk, 2-(Z-amino)-3,5-dimethylbenzoyl-Asp-fmk, 2-(Z-amino)-4-chlorobenzoyl-Asp-fmk, 2-(Z-amino)-5-chlorobenzoyl-Asp-fmk, 2-(Z-amino)-5-fluorobenzoyl-Asp-fmk, 2-(Z-amino)-6-fluorobenzoyl-Asp-fmk, cis-2-(Z-amino)cyclohexanecarboxyl-Asp-fmk, 2-(Z-amino)-5-methylbenzoyl-Asp-fmk, 2-(Z-amino)-6-methylbenzoyl-Asp-fmk, 2-(Z-amino)-6-chlorobenzoyl-Asp-fmk, 2-(Z-amino)-3-methoxybenzoyl-Asp-fmk, 2-(Z-amino)thiophene-2-carboxyl-Asp-fmk, 2-(methoxycarbonylamino)thiophene-2-carboxyl -Asp-fmk, cis-2-(Z-amino)cyclopentanecarboxyl-Asp-fmk, trans-2-(Z-amino)cyclopentanecarboxyl-Asp-fmk, 2-(Z-amino)benzoyl-Asp-DCB-methylketone, methoxycarbonyl-Val-(2-aminobenzoyl)-Asp-fmk, Z-Glu-(2-aminobenzoyl)-Asp-fmk, and Z-Val-(2-aminobenzoyl)-Asp-fmk.

In another preferred embodiment, the caspase inhibitor has the formula VI: 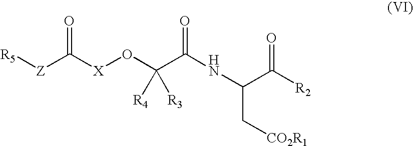
or a pharmaceutically acceptable salt thereof;
wherein R₁ is an optionally substituted alkyl or hydrogen;
R₂ is hydrogen or optionally substituted alkyl;
R₃ and R₄ independently are hydrogen, optionally substituted aryl, optionally substituted heterocyclic, optionally substituted carbocyclic, optionally substituted heteroaryl, optionally substituted alkyl, optionally substituted alkenyl, or optionally substituted alkynyl;
R₅ is an optionally substituted alkyl, optionally substituted carbocyclic, optionally substituted heterocyclic, optionally substituted aryl or optionally substituted heteroaryl;
Z is O, S, NR₈, or (CR₉R₁₀)ₙ, where R₈, R₉ and R₁₀ independently are hydrogen, alkyl or cycloalkyl, and n is 0, 1, 2, or 3; and
X is a peptide of 1-2 amino acids or a bond. Where X is one amino acid, it may be any one of the common 20 amino acids e.g., Ala, Val, Leu, Ile, Pro, Phe, Trp, Met, Gly, Ser, Thr, Cys, Tyr, Asp, Asn, Glu, Asn, Lys, Arg and His. Where X is a peptide, it may be Asp-Glu, Asp-Ala, Asp-Phe, Val-Glu, Leu-Glu, Thr-Glu, Ile-Glu, Tyr-Glu, and Trp-Glu.

Examples of such caspase inhibitors include 1-(Carbonyl-Asp-CH₂F)ethyl N-phenylcarbamate, 1-(Carbonyl-Asp-CH₂F)ethyl N-benzylcarbamate, 2-Methyl-1-(carbonyl-Asp-CH₂F)propyl N-phenylcarbamate, 2-Methyl-1-(carbonyl-Asp-CH₂F)propyl N-benzylcarbamate, 2-Methyl-1-(carbonyl-Asp-CH₂F)propyl N-(2,6-dichlorophenyl)carbamate, 2-Methyl-1-(carbonyl-Asp-CH₂F)propyl N-(2,5-dichlorophenyl)-carbamate, 2-Methyl-1-(carbonyl-Asp-CH₂F)propyl N-(2,4-dichlorophenyl)-carbamate, 2-Methyl-1-(carbonyl-Asp-CH₂DCB)propyl N-phenylcarbamate, 2-Methyl-1-(carbonyl-Asp-CH₂DCB)propyl N-(2,6-dichlorophenyl)-carbamate, 2-Methyl-1-(carbonyl-Asp-CH₂PTP)propyl N-phenylcarbamate, 2-Methyl-1-(carbonyl-Asp-CH₂PTP)propyl N-(2,6-dichlorophenyl)-carbamate, 2-Methyl-1-(carbonyl-Asp-CH₂DPP)propyl N-phenylcarbamate, 2-Methyl-1-(carbonyl-Asp-CH₂DPP)propyIN-(2,6-dichlorophenyl)-carbamate, 2-Methyl-1-(carbonyl-Asp-CH₂F)propyl N-(2-methyl-1-methoxycarbonyl-propyl)carbamate, 2-Methyl-1-(carbonyl-Asp-CH₂F)propyl N-(3-fluorophenyl)carbamate, 2-Methyl-1-(carbonyl-Asp-CH₂F)propyl N-(4-fluorophenyl)carbamate, 2-Methyl-1-(carbonyl-Asp-CH₂F)propyl N-(3,4-difluorophenyl)carbamate, 2-Methyl-1-(carbonyl-Asp-CH₂F)propyl N-(4-phenoxyphenyl)carbamate, 1-(Carbonyl-Asp-CH₂F)propyl N-phenylcarbamate, 1-(Carbonyl-Asp-CH₂F)butyl N-phenylcarbamate, 1-(Carbonyl-Asp-CH₂F)-2-propenyl N-phenylcarbamate, 2-(4-Imidazolyl)-1-(carbonyl-Asp-CH₂F)ethyl N-phenylcarbamate, 2-Phenyl-1-(carbonyl-Asp-CH₂F)ethyl N-phenylcarbamate, 2-Methyl-1-(carbonyl-Asp-CH₂F)butyl N-phenylcarbamate, 3-Methyl-1-(carbonyl-Asp-CH₂F)butyl N-phenylcarbamate, 1-Phenyl-1-(carbonyl-Asp-CH₂F)methyl N-phenylcarbamate, 1-(2-Chlorophenyl)-1-(carbonyl-Asp-CH₂F)methyl N-phenylcarbamate, 1-(4-Chlorophenyl)-1-(carbonyl-Asp-CH₂F)methyl N-phenylcarbamate, 1-Cyclohexyl-1-(carbonyl-Asp-CH₂F)methyl N-phenylcarbamate, 2-Chloro-1-(carbonyl-Asp-CH₂F)ethyl N-phenylcarbamate, 2,2,2-Trifluoro-1-(carbonyl-Asp-CH₂F)ethyl N-phenylcarbamate, and Z-Valine 2-methyl-1-(carbonyl-Asp-CH₂F)propyl ester.

In another preferred embodiment, the caspase inhibitor has the formula VII: 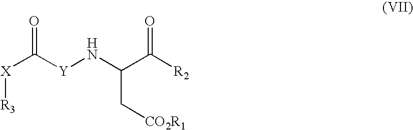
or a pharmaceutically acceptable salt thereof;
wherein R₁ is an optionally substituted alkyl or hydrogen;
R₂ is hydrogen or optionally substituted alkyl;
R₃ is an alkyl, saturated carbocyclic, partially saturated carbocyclic, aryl, saturated heterocyclic, partially saturated heterocyclic or heteroaryl group, wherein said group is optionally substituted;
X is O, S, NR₄, or (CR₄R₅)ₙ, where R₄ and R₅ are, at each occurrence, independently selected from the group consisting of hydrogen, alkyl and cycloalkyl, and n is 0, 1, 2, or 3; or
X is NR₄, and R₃ and R₄ are taken together with the nitrogen atom to which they are attached to form a saturated heterocyclic, partially saturated heterocyclic or heteroaryl group, wherein said group is optionally substituted; or
X is CR₄R₅, and R₃ and R₄ are taken together with the carbon atom to which they are attached to form a saturated carbocyclic, partially saturated carbocyclic, aryl, saturated heterocyclic, partially saturated heterocyclic or oxygen-containing heteroaryl group, wherein said group is optionally substituted; and
Y is a residue of a natural or non-natural amino acid; provided that when X is O, then R₃ is not unsubstituted benzyl or t-butyl; and when X is CH₂, then R₃ is not hydrogen.

Examples of such caspase inhibitors include 2-Chlorobenzyloxycarbonyl-Val-Asp-fmk, 3-Chlorobenzyloxycarbonyl-Val-Asp-fmk, 4-Chlorobenzyloxycarbonyl-Val-Asp-fmk, Phenethoxycarbonyl-Val-Asp-fmk, Cyclohexylmethoxycarbonyl-Val-Asp-fmk, Methoxycarbonyl-Val-Asp-fmk, Ethoxycarbonyl-Val-Asp-fmk, Isopropyloxycarbonyl-Val-Asp-fmk, 2-Chlorobenzyloxycarbonyl-Ile-Asp-fmk, 3-Chlorobenzyloxycarbonyl-Ile-Asp-fmk, 4-Chlorobenzyloxycarbonyl-Ile-Asp-fmk, Phenylacetyl-Val-Asp-fmk, 4-Nitrobenzyloxycarbonyl-Val-Asp-fmk, 2,5-Dimethylbenzyloxycarbonyl-Val-Asp-fmk, 3,4-Dichlorobenzyloxycarbonyl-Val-Asp-fmk, 3,5-Dichlorobenzyloxycarbonyl-Val-Asp-fmk, 2,5-Dichlorobenzyloxycarbonyl-Val-Asp-fmk, 2,6-Dichlorobenzyloxycarbonyl-Val-Asp-fmk, 2,4-Dichlorobenzyloxycarbonyl-Val-Asp-fmk, 2,4-Dimethylbenzyloxycarbonyl-Val-Asp-fmk, 4-Ethylbenzyloxycarbonyl-Val-Asp-fmk, 4-Bromobenzyloxycarbonyl-Val-Asp-fmk, 4-Fluorobenzyloxycarbonyl-Val-Asp-fmk, Cyclopentylmethoxycarbonyl-Val-Asp-fmk, 4-Trifluoromethylbenzyloxycarbonyl-Val-Asp-fmk, 3-Phenylpropionyl-Val-Asp-fmk, Benzylaminocarbonyl-Val-Asp-fmk, 3-Phenylpropyloxycarbonyl-Val-Asp-fmk, 2,4-Difluorobenzyloxycarbonyl-Val-Asp-fmk, 3,4-Difluorobenzyloxycarbonyl-Val-Asp-fmk, 4-Morpholinecarbonyl-Val-Asp-fmk, 4-Pyridylmethoxycarbonyl-Val-Asp-fmk, 2-Pyridylmethoxycarbonyl-Val-Asp-fmk, 2,6-Dichlorobenzyloxycarbonyl-Val-Asp-DCB-methylketone, Isobutoxycarbonyl-Val-Asp-fmk, Propionyl-Val-Asp-fmk, Benzyl-glutaryl-Val-Asp-fmk,Glutaryl-Val-Asp-fmk, 3-(2-Phenyloxyphenyl)propionyl-Val-Asp-fmk, 3-(5-Bromo-2-hydroxyphenyl)propionyl-Val-Asp-fmk, 3-Fluorobenzyloxycarbonyl-Val-Asp-fmk, 2-Fluorobenzyloxycarbonyl-Val-Asp-fmk, 3-Methylbenzyloxycarbonyl-Val-Asp-fmk, 2-Chloro-4-fluorobenzyloxycarbonyl-Val-Asp-fmk, 2-Naphthylmethoxycarbonyl-Val-Asp-fmk, p-Toluenesulfonyl-Val-Asp-fmk, and p-Toluenesulfonyl-Phe-Asp-fmk.

Other caspase inhibitors that may be used in the practice of the invention include without limitation those described in WO 93/05071, WO 93/09135, WO 93/14777, WO 95/26958, WO 95/29672, WO 95/33751, WO 96/03982, WO 96/30395, WO 97/07805, WO 97/08174, WO 97/22618, WO 97/27220, WO 98/11109, WO 98/11129, WO 98/16502, WO 98/16504, WO 98/16505, WO 98/24804, WO 98/24805, WO 99/46248, WO 99/47545, WO 00/09664, WO 00/32620, WO 00/55127, WO 00/59536, WO 01/10383, WO 01/21599, WO 01/21600, WO 01/27140, WO 01/39792, WO 01/42216, WO 01/58526, WO 01/60400, WO 01/72707, WO 01/90070, WO 01/94351, EP 519748, EP 547699, EP 618223, EP 623592, EP623606, EP 628550, EP 644198, EP 1076563, EP 1095018, EP 1135406, EP 1163214, EP 1165490, EP 1169056, EP 1177168, U.S. Pat. No. 5,430,128, U.S. Pat. No. 5,434,248, U.S. Pat. No. 5,462,939, U.S. Pat. No. 5,552,400, U.S. Pat. No. 5,565,430, U.S. Pat. No. 5,585,357, U.S. Pat. No. 5,585,486, U.S. Pat. No. 5,622,967, U.S. Pat. No. 5,639,745, U.S. Pat. No. 5,656,627, U.S. Pat. No. 5,670,494, U.S. Pat. No. 5,677,283, U.S. Pat. No. 5,716,929, U.S. Pat. No. 5,739,279, U.S. Pat. No. 5,756,465, U.S. Pat. No. 5,756,466, U.S. Pat. No. 5,798,247, U.S. Pat. No. 5,798,442, U.S. Pat. No. 5,834,514, U.S. Pat. No. 5,843,904, U.S. Pat. No. 5,843,905, U.S. Pat. No. 5,847,135, U.S. Pat. No. 5,866,545, U.S. Pat. No. 5,869,519, U.S. Pat. No. 5,874,424, U.S. Pat. No. 5,932,549, U.S. Pat. No. 6,004,933, U.S. Pat. No. 6,045,990, U.S. Pat. No. 6,057,333, 6,200,969, U.S. Pat. No. 6,218,419, U.S. Pat. No. 6,225,288, Mjalli et al., Bioorg. Med. Chem. Lett. 3:2689-2693 (1993), Mjalli et al., Bioorg. Med. Chem. Lett. 4:1965-1968 (1994), Mjalli et al., Bioorg. Med. Chem. Lett. 5:1405-1408 (1995), Mjalli et al., Bioorg. Med. Chem. Lett. 5:1409-1414 (1995), Thornberry et al., Biochem. 33.3934-3940 (1994), Dolle et al., J. Med. Chem. 37: 563-564 (1994), Dolle et al., J. Med. Chem. 37: 3863-3866 (1994), Dolle et al., J. Med. Chem. 38: 220-222 (1995), Graybill et al., Bioorg. Med. Chem. Lett. 7:41-46 (1997), Semple et al., Bioorg. Med. Chem. Lett. 8:959-964 (1998), and Okamoto et al., Chem. Pharm. Bull. 47:11-21 (1999).

With regard to the caspase inhibitors described herein, useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups. Also contemplated is a trimethylene group substituted on two adjoining positions on the benzene ring of the compounds of the invention.

Optional substituents include one or more alkyl; halo; haloalkyl; cycloalkyl; aryl optionally substituted with one or more lower alkyl, halo, haloalkyl or heteroaryl groups; aryloxy optionally substituted with one or more lower alkyl, halo, haloalkyl or heteroaryl groups; aralkyl; heteroaryl optionally substituted with one or more lower alkyl, haloalkyl and aryl groups; heteroaryloxy optionally substituted with one or more lower alkyl, haloalkyl and aryl groups; alkoxy; alkylthio; arylthio; amino; acyloxy; arylacyloxy optionally substituted with one or more lower alkyl, halo alkyl and aryl groups; diphenylphosphinyloxy optionally substituted with one or more lower alkyl, halo or haloalkyl groups; heterocyclo optionally substituted with one or more lower alkyl, haloalkyl and aryl groups; heterocycloalkyloxy optionally substituted with one or more lower alkyl, haloalkyl and aryl groups; partially unsaturated heterocycloalkyl optionally substituted with one or more lower alkyl, haloalkyl and aryl groups; or partially unsaturated heterocycloalkyloxy optionally substituted with one or more lower alkyl, haloalkyl and aryl groups.

Useful aryl groups are C₆₋₁₄ aryl, especially C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as defined above, as well as cycloalkenyl groups, such as cyclopentenyl, cycloheptenyl and cyclooctenyl.

Useful halo or halogen groups include fluorine, chlorine, bromine and iodine.

Useful arylalkyl groups include any of the above-mentioned C₁₋₁₀ alkyl groups substituted by any of the above-mentioned C₆₋₁₄ aryl groups. Useful values include benzyl, phenethyl and naphthylmethyl.

Useful haloalkyl groups include C₁₋₁₀ alkyl groups substituted by one or more fluorine, chlorine, bromine or iodine atoms, e.g. fluoromethyl, difluoromethyl, trifluoromethyl, pentafluoroethyl, 1,1-difluoroethyl, chloromethyl, chlorofluoromethyl and trichloromethyl groups.

Useful alkoxy groups include oxygen substituted by one of the C₁₋₁₀ alkyl groups mentioned above.

Useful alkylthio groups include sulphur substituted by one of the C₁₋₁₀ alkyl groups mentioned above. Also included are the sulfoxides and sulfones of such alkylthio groups.

Useful acylamino groups are any C₁₋₆ acyl (alkanoyl) attached to an amino nitrogen, e.g. acetamido, propionamido, butanoylamido, pentanoylamido, hexanoylamido as well as aryl-substituted C₂₋₆ substituted acyl groups.

Useful acyloxy groups are any C₁₋₆ acyl (alkanoyl) attached to an oxy (—O—) group, e.g. formyloxy, acetoxy, propionoyloxy, butanoyloxy, pentanoyloxy, hexanoyloxy and the like.

Useful arylacyloxy groups include any of the aryl groups mentioned above substituted on any of the acyloxy groups mentioned above, e.g. 2,6-dichlorobenzoyloxy, 2,6-difluorobenzoyloxy and 2,6-di-(trifluoromethyl)-benzoyloxy groups.

Useful amino groups include —NH₂, —NHR₁₁, and —NR₁₁R₁₂, wherein R₁₁ and R₁₂ are C₁₋₁₀ alkyl or cycloalkyl groups as defined above.

Useful saturated or partially saturated heterocyclic groups include tetrahydrofuranyl, pyranyl, piperidinyl, piperizinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

Useful heteroaryl groups include any one of the following: thienyl, benzo[b]thienyl, naphtho[2,3-b]thienyl, thianthrenyl, furyl, pyranyl, isobenzofuranyl, chromenyl, xanthenyl, phenoxanthiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g. a pyridyl N-oxide, pyrazinyl N-oxide, pyrimidinyl N-oxide and the like.

Preferred N-terminal protecting groups include t-butyloxycarbonyl, acetyl and benzyloxycarbonyl.

Amino acids include any of the naturally occurring amino acids such as the L-forms of tyrosine, glycine, phenylalanine, methionine, alanine, serine, isoleucine, leucine, threonine, valine, proline, lysine, histidine, glutamine, glutamic acid, tryptophan, arginine, aspartic acid, asparagine and cysteine. Examples of non-natural amino acids include, without limitation, the enantiomeric and racemic forms of 2-methylvaline, 2-methylalanine, (2-i-propyl)-β-alanine, phenylglycine, 4-methylphenylglycine, 4-isopropylphenylglycine, 3-bromophenylglycine, 4-bromophenylglycine, 4-chlorophenylglycine, 4-methoxyphenylglycine, 4-ethoxyphenylglycine, 4-hydroxyphenylglycine, 3-hydroxyphenylglycine, 3,4-dihydroxyphenylglycine, 3,5-dihydroxyphenylglycine, 2,5-dihydrophenylglycine, 2-fluorophenylglycine, 3-fluorophenylglycine, 4-fluorophenylglycine, 2,3-difluorophenylglycine, 2,4-difluorophenylglycine, 2,5-difluorophenylglycine, 2,6-difluorophenylglycine, 3,4-difluorophenylglycine, 3,5-difluorophenylglycine, 2-(trifluoromethyl)phenylglycine, 3-(trifluoromethyl)phenylglycine, 4-(trifluoromethyl)phenylglycine, 2-(2-thienyl)glycine, 2-(3-thienyl)glycine, 2-(2-furyl)glycine, 3-pyridylglycine, 4-fluorophenylalanine, 4-chlorophenylalanine, 2-bromophenylalanine, 3-bromophenylalanine, 4-bromophenylalanine, 2-naphthylalanine, 3-(2-quinoyl)alanine, 3-(9-anthracenyl)alanine, 2-amino-3-phenylbutanoic acid, 3-chlorophenylalanine, 3-(2-thienyl)alanine, 3-(3-thienyl)alanine, 3-phenylserine, 3-(2-pyridyl)serine, 3-(3-pyridyl)serine, 3-(4-pyridyl)serine, 3-(2-thienyl)serine, 3-(2-furyl)serine, 3-(2-thiazolyl)alanine, 3-(4-thiazolyl)alanine, 3-(1,2,4-triazol-1-yl)-alanine, 3-(1,2,4-triazol-3-yl)-alanine, hexafluorovaline, 4,4,4-trifluorovaline, 3-fluorovaline, 5,5,5-trifluoroleucine, 2-amino-4,4,4-trifluorobutyric acid, 3-chloroalanine, 3-fluoroalanine, 2-amino-3-flurobutyric acid, 3-fluoronorleucine, 4,4,4-trifluorothreonine, L-allylglycine, tert-Leucine, propargylglycine, vinylglycine, S-methylcysteine, cyclopentylglycine, cyclohexylglycine, 3-hydroxynorvaline, 4-azaleucine, 3-hydroxyleucine, 2-amino-3-hydroxy-3-methylbutanoic acid, 4-thiaisoleucine, acivicin, ibotenic acid, quisqalic acid, 2-indanylglycine, 2-aminoisobutyric acid, 2-cyclobutyl-2-phenylglycine, 2-isopropyl-2-phenylglycine, 2-methylvaline, 2,2-diphenylglycine, 1-amino-1-cyclopropanecarboxylic acid, 1-amino-1-cyclopentanecarboxylic acid, 1-amino-1-cyclohexanecarboxylic acid, 3-amino-4,4,4-trifluorobutyric acid, 3-phenylisoserine, 3-amino-2-hydroxy-5-methylhexanoic acid, 3-amino-2-hydroxy-4-phenylbutyric acid, 3-amino-3-(4-bromophenyl)propionic acid, 3-amino-3-(4-chlorophenyl)propionic acid, 3-amino-3-(4-methoxyphenyl)propionic acid, 3-amino-3-(4-fluorophenyl)propionic acid, 3-amino-3-(2-fluorophenyl)propionic acid, 3-amino-3-(4-nitrophenyl)propionic acid, and 3-amino-3-(1-naphthyl)propionic acid.

Useful electronegative leaving groups include F, Cl, TsO—, MeO—, ArO—, ArCOO—, ArN—, and ArS—.

Certain of the compounds may exist as stereoisomers including optical isomers. The invention includes the use of all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts such as hydrochloride, hydrobromide, phosphate, sulphate, citrate, lactate, tartrate, maleate, fumarate, mandelate and oxalate; and inorganic and organic base addition salts with bases such as sodium hydroxy, potassium hydroxy and Tris(hydroxymethyl)aminomethane (TRIS, tromethane).

Examples of prodrugs that may be used include compounds having substituted alkyl group such as CH₂OCH₃ and CH₂OCOCH₃ (AM ester).

The caspase inhibitors may be prepared according to methods well known in the art and by those methods in the publications, patent applications and patents cited herein.

The caspase inhibitors may be administered as part of a pharmaceutical composition comprising a pharmaceutically acceptable carrier, wherein the caspase inhibitors are present in an amount which is effective to achieve its intended purpose. While individual needs vary, determination of optimal ranges of effective amounts of each component is within the skill of the art.

Typically, the compounds may be administered to mammals, e.g. humans, orally at a dose of 0.0025 to 50 mg/kg, or an equivalent amount of the pharmaceutically acceptable salt thereof, per day of the body weight of the mammal being treated. Preferably, about 0.01 to about 10 mg/kg is orally administered. For intramuscular injection, the dose is generally about one-half of the oral dose, e.g. about 0.0025 to about 25 mg/kg, and most preferably, from about 0.01 to about 5 mg/kg.

The unit oral dose may comprise from about 0.01 to about 50 mg, preferably about 0.1 to about 10 mg of the compound. The unit dose may be administered one or more times daily as one or more tablets each containing from about 0.1 to about 10 mg, conveniently about 0.25 to 50 mg of the compound or its solvates.

In a topical formulation, the compound may be present at a concentration of about 0.01 to 100 mg per gram of carrier. In a preferred embodiment, the compound is present at a concentration of about 0.07-1.0 mg/ml, more preferably, about 0.1 to 0.5 mg/ml, most preferably, about 0.4 mg/ml.

For veterinary uses, higher levels may be administered as necessary.

Suitable pharmaceutically acceptable carriers comprise excipients and auxiliaries which facilitate processing of the compounds into preparations which can be used pharmaceutically. Preferably, the preparations, particularly those preparations which can be administered orally or topically and which can be used for the preferred type of administration, such as tablets, dragees, slow release lozenges and capsules, mouth rinses and mouth washes, gels, liquid suspensions, hair rinses, hair gels, shampoos and also preparations which can be administered rectally, such as enemas and suppositories, as well as suitable solutions for administration by injection, topically or orally, contain from about 0.01 to 99 percent, preferably from about 0.25 to 75 percent of active compound(s), together with the excipient.

Also included within the scope of the present invention are the use of non-toxic pharmaceutically acceptable salts of the caspase inhibitors. Acid addition salts are formed by mixing a solution of the particular caspase inhibitor with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the particular caspase inhibitor with a solution of a pharmaceutically acceptable non-toxic base such as sodium hydroxide, potassium hydroxide, choline hydroxide, sodium carbonate, Tris and the like.

The caspase inhibitors may be administered to any animal which may experience the beneficial effects of the invention. Foremost among such animals are mammals, e.g., humans, although the invention is not intended to be so limited.

The caspase inhibitors and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired. In general, the caspase inhibitors may be administered locally to the tissues that are to be protected from apoptosis. For example, the caspase inhibitors may be administered locally to treat, ameliorate, or prevent apoptotic cell death in the mouth or gastrointestinal tract, such as a mouth wash for the treatment of oral mucositis resulting from ingestion of radionuclides; and IV injectable aqueous solution for the treatment of bone marrow, immune system, neuronal or liver cell death; and an oral formulation suitable for coating the gastrointestinal surfaces or an enema or suppository formulation for the treatment of gastrointestinal mucositis including proctitis. The caspase inhibitors may also be applied through a bladder catheter for the treatment, amelioration or prevention of bladder mucositis resulting from radionuclide exposure. Alternatively or concurrently, the caspase inhibitors may be applied topically to the skin and/or scalp to treat, ameliorate or prevent apoptotic cell death of hair and skin cells. In another embodiment, the caspase inhibitors may be administered to tissues surrounding a site which is to be treated with a radiopharmaceutical agent for the purpose of cytoprotection. For example, when a cytotoxic radiopharmaceutical agent is to be administered to a tumor as a method of treatment, the normal cells surrounding the tumor can be protected from the cytotoxic agent by administering caspase inhibitors to the tissues surrounding the tumor. This embodiment is applicable to the treatment of blood vessels with cytotoxic radiopharmaceutical agents to prevent restenosis as well as the treatment of other forms of undesirable cell proliferation. In a further embodiment, the caspase inhibitors may be administered systemically, e.g. by i.v. injection, to treat, ameliorate or prevent apoptotic cell death of the gastrointestinal tract cells, mouth epithelial cells, bone marrow cells, skin cells and hair cells. Importantly, the caspase inhibitor can be applied prior to exposure to the radionuclides, biological agents, or chemical agents, thus preventing the onset of the damaging effects thereof.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered.

Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances which increase the viscosity of the suspension and include, for example, sodium carboxymethyl cellulose, sorbitol, and/or dextran. Optionally, the suspension may also contain stabilizers.

One or more additional substances which have beneficial effects on the treated cells may also be incorporated in the compositions. Thus, the composition may also contain one or more compounds capable of increasing cyclic-AMP levels in the skin. Suitable compounds include adenosine or a nucleic acid hydrolysate in an amount of about 0.1-1% and papaverine, in an amount of about 0.5-5%, both by weight based on the weight of the composition. Also suitable are β-adrenergic agonists such as isoproterenol, in an amount of about 0.1-2% or cyclic-AMP, in an amount of about 0.1-1%, again both by weight based on the weight of the composition. Other suitable types of additional active ingredients which may be incorporated in the pharmaceutical compositions include any compounds known to have a beneficial effect on skin. Such compounds include retinoids such as Vitamin A, in an amount of about 0.003-0.3% by weight and chromanols such as Vitamin E or a derivative thereof in an amount of about 0.1-10% by weight, both based on the weight of the composition. Additionally, anti-inflammatory agents and keratoplastic agents may be incorporated in the pharmaceutical compositions. A typical anti-inflammatory agent is a corticosteroid such as hydrocortisone or its acetate in an amount of about 0.25-5% by weight, or a corticosteroid such as dexamethasone in an amount of about 0.025-0.5% by weight, both based on the weight of the composition. A typical keratoplastic agent that may be included in a topical composition for the skin is coal tar in an amount of about 0.1-20% or anthralin in an amount of about 0.05-2% by weight, both based on the weight of the composition.

The topical compositions may be formulated preferably as oils, creams, lotions, ointments and the like by choice of appropriate carriers.

Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the caspase inhibitor, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the caspase inhibitor in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the caspase inhibitor, in a suitable high molecular weight alcohol such as propylene glycol or polyethylene glycol.

In addition, these compositions may include other medicinal agents, growth factors, wound sealants, carriers, etc., that are known or apparent to those skilled in the art.

In a preferred embodiment, the caspase inhibitor is formulated as part of a mouthwash for the treatment, amelioration or prevention of oral mucositis resulting from ingestion of radionuclides, biological agents, or chemical agents. Such mouthwashes are aqueous solutions of the caspase inhibitor which may also contain alcohol, glycerin, synthetic sweeteners and surface-active, flavoring and coloring agents. They may also contain anti-infective agents such as hexetidine and cetylpyridinium chloride. The mouthwashes may also contain topical anesthetics (e.g. benzocaine, cocaine, dyclonine hydrochloride, lidocaine, proparacaine hydrochloride or teracaine hydrochloride), for example, for relieving pain of radiation-induced sores.

The mouth washes may have either acidic or basic pH. See Remington's Pharmaceutical Sciences, A. R. Gennaro (ed.), Mack Publishing Company, pp. 1045, 1046, 1526 and 1965 (1990).

In another preferred embodiment, the caspase inhibitor is formulated as an oral formulation which is capable of coating the gastrointestinal surfaces for the treatment, amelioration or prevention of gastrointestinal mucositis resulting from exposure to radionuclides, biological agents, or chemical agents and, in particular, injection of radionuclides, biological agents, or chemical agents. Examples of gastrointestinal mucositis include esophageal mucositis, gastric mucositis, and intestinal mucositis. Such formulations may comprise gastric antacids such as aluminum carbonate, aluminum hydroxide gel, bismuth subnitrate, bismuth subsalicylate, calcium carbonate, dihydroxyaluminum sodium carbonate, magaldrate, magnesium carbonate, magnesium hydroxide, magnesium oxide, sodium bicarbonate, milk of bismuth, dihydroxyaluminum aminoacetate, magnesium phosphate, magnesium trisilicate and mixtures thereof. Other additives include without limitation H₂-receptor antagonists, digestants, anti-emetics, adsorbants, and miscellaneous agents. See Remington's Pharmaceutical Sciences, A. R. Gennaro (ed.), Mack Publishing Company, pp. 774-778 (1990).

Exposure to radiation, biological agents, or chemical agents often induces early and late onset emesis in a subject. Thus, in one embodiment an antiemetic is coadminstered together with the caspase inhibitor to avoid emesis and retain contact of the caspase inhibitor with the gastrointestinal tract. Examples of such antiemetics include without limitation compounds that block the dopaminergic emetic receptors such as metoclopramide and trimethobenzamide, and cannabinoids. Metoclopramide may be administered orally prior to and/or during radionuclides exposure to prevent the early emesis response and then later by intranasal administration according to U.S. Pat. Nos. 5,760,086 and 4,536,386 to prevent delayed onset emesis. During the period after exposure to radionuclide, both the caspase inhibitor and the antiemetic may be coadministered to treat, ameliorate or prevent gastrointestinal mucositis.

In a further embodiment, the caspase inhibitor may be formulated as an IV injectable solution for the treatment, amelioration or prevention of bone marrow, immune system, neuronal or liver cell death due to exposure to radionuclides, biological agents, or chemical agents.

The compositions may be administered to a warm-blooded animal, such as human, already suffering from radionuclide, biological agent, or chemical agent exposure-induced cell death, or, more preferably, before or during exposure to radionuclides, biological agents, or chemical agents.

### EXAMPLE 1

### Caspase Inhibitor Cbz-Val-Asp-CH₂F Is Effective in Protecting Mice from Death Caused by Exposure to Gamma Radiation

Experiment A

This study was conducted to determine a lethal Gamma radiation dose in mice.

Materials:

48 ICR Mice (CD-1), male or female, body weight of 20-30 g.

Procedure:

All the mice were divided into 8 group (6 mice in each group, 3 males and 3 females) and received a single dose of gamma radiation at La Jolla Institute of Immunology (Gammacell 40, Low Dose Rate Laboratory Irradiator by Nordian International, Inc. with Cs-137 as radiation source) as indicated in Table 1.

The animals were weighed, observed and survival data were collected for 3 weeks. Animal death were observed from day 8 to day 12 following Gamma irradiation at the high doses. Table 1 summarized the data at day 24.

TABLE 1
Death of Mice Caused by Exposure to Gamma Irradiation

| Group # | Radiation dose (Rad) | Dead/Total animals |
|---|---|---|
| 1 | 400 | 0/6 |
| 2 | 500 | 0/6 |
| 3 | 600 | 1/6 |
| 4 | 700 | 3/6 |
| 5 | 800 | 5/6 |
| 6 | 900 | 6/6 |
| 7 | 1000 | 6/6 |
| 8 | 0 | 0/6 |

The results showed that 100% lethality was observed at day 8 to 12 following radiation at 900 to 1000 Rad. 85% and 50% lethality within the same time period was observed at 800 and 700 Rad, respectively. It was concluded from this study that dose of ≧850 Rad would result in 100% lethality in this model.

Experiment B

This study was conducted to determine the effects of caspase inhibitor Cbz-Val-Asp-CH₂F to protect mice from death caused by exposure to gamma radiation.

Materials:

80 ICR Mice (CD-1), male or female, body weight of 20-30 g.

Procedure:

All the mice were divided into 10 groups (8 mice in each group, 4 males and 4 females) and received a single dose of gamma radiation of 850 or 950 Rad at La Jolla Institute of Immunology (Gammacell 40, Low Dose Rate Laboratory Irradiator by Nordian International, Inc. with Cs-137 as radiation source) as indicated in Table 2.

Cbz-Val-Asp-CH₂F was dissolved in 0.05 M of tris-base aqueous solution at a concentration of 10 mg/mL and administered by IV injection to the mice. Mice were treated with a single injection of 10 mg/kg, 20 mg/kg or 50 mg of Cbz-Val-Asp-CH₂F 10 min following irradiation. Mice in control groups 4 and 9 were injected with vehicle.

The animals were weighed, observed and survival data were collected for 3 weeks. Animal death were observed from day 8 to day 12 following Gamma irradiation. Table 2 summarized the data at day 25.

TABLE 2
Effects of Caspase Inhibitor Cbz-Val-Asp-CH₂F in Protecting Mice
from Death Caused by Exposure to Gamma Radiation

| | Radiation dose | Treatment dose | Dead/Total |
|---|---|---|---|
| Group # | (Rad) | (mg/kg) | animals |
| 1 | 850 | 10 | 8/8 |
| 2 | 850 | 20 | 5/8 |
| 3 | 850 | 50 | 5/8 |
| 4 | 850 | 0 | 8/8 |
| 5 | 0 | 0 | 0/8 |
| 6 | 950 | 10 | 8/8 |
| 7 | 950 | 20 | 8/8 |
| 8 | 950 | 50 | 8/8 |
| 9 | 950 | 0 | 8/8 |
| 10 | 0 | 0 | 0/8 |

The results from this study showed that when mice was irradiated with 850 Rad of Gamma radiation, a single IV administration of 20 mg/kg and 50 mg/kg of Cbz-Val-Asp-CH₂F protected about 38% of the animals from death caused by exposure to radiation. In comparison, there was 100% lethality in the control group received 850 Rad of Gamma radiation. This indicates that the caspase inhibitor Cbz-Val-Asp-CH₂F is effective in protecting the mice from death caused by radiation. In the study with 950 Rad all animals died, suggesting that Cbz-Val-Asp-CH₂F is not effective with higher dose of radiation.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1. A method of treating, ameliorating or preventing a disease or condition caused by exposure to radionuclides, biological agents, or chemical agents in an animal, comprising administering to an animal in need thereof an effective amount of a caspase inhibitor such that cell death in response to said exposure to said radionuclides, biological agents, or chemical agents is inhibited.

2. The method of claim 1, wherein said cell death occurs in cells of the gastrointestinal tract, skin, hair, bone marrow, immune system, nervous system or liver.

3. The method of claim 1, wherein said caspase inhibitor is administered topically or orally.

4. The method of claim 1, wherein said caspase inhibitor is administered systemically by intravenous, intraperitoneal, intramuscular, or subcutaneous injection.

5. The method of claim 1, wherein said caspase inhibitor is administered as part of a pharmaceutical composition comprising a pharmaceutically acceptable carrier.

6. The method of claim 1, wherein said exposure to radionuclides, biological agents, or chemical agents is unintentional.

7. The method of claim 6, wherein said radionuclides, biological agents, or chemical agents are from a nuclear power plant, manufacturing or processing plant, research facility, or hospital.

8. The method of claim 1, wherein said exposure to radionuclides, biological agents, or chemical agents is intentional.

9. The method of claim 8, wherein said radionuclides, biological agents, or chemical agents are from a spill or a bomb.

10. The method of claim 1, wherein said radionuclides are part of a radiopharmaceutical agent.

11. The method of claim 1, wherein said radionuclides are selected from the group consisting of actinium (²²⁵Ac), americium (²⁴¹Am), antimony (¹²⁴Sb, ¹²⁵Sb), arsenic (⁷²As, ⁷³As, ⁷⁴As), astatine (²¹¹At), barium (¹⁰³Ba, ¹⁴⁰Ba), beryllium (⁷Be), bismuth (²⁰⁶Bi, ²⁰⁷Bi, ²¹²Bi, ²¹³Bi), bromine (⁷⁷Br), cadmium (¹⁰⁹Cd, ¹¹⁵Cd), calcium (⁴⁵Ca), carbon (¹⁴C), cerium (¹³⁹Ce, ¹⁴¹Ce, ¹⁴⁴Ce), cesium (¹²⁹Cs, ¹³⁷Cs), chromium (⁵¹Cr, ⁵⁶Cr), cobalt (⁵⁵Co, ⁵⁶Co, ⁵⁷Co, ⁵⁸Co, ⁶⁰Co, ⁶⁴Co), copper (⁶¹Cu, ⁶⁴Cu, ⁶⁷Cu), erbium (¹⁶⁹Er), europium (¹⁵²Eu), fluorine (¹⁸F), gadolinium (¹⁵³Gd), gallium (⁶⁷Ga, ⁶⁸Ga), gold (¹⁹⁵Au, ¹⁹⁸Au, ¹⁹⁹Au), hafnium (¹⁷⁵Hf, ¹⁸¹Hf), holmium (¹⁶⁶Ho), hydrogen (³H), krypton (⁸⁵Kr), iodine (¹²³I, ¹²⁵I, ¹²⁶I, ¹³¹I, ¹³³I), indium (¹¹¹In, ¹¹³In), iridium (¹⁹²Ir), iron (⁵²Fe, ⁵⁵Fe, ⁵⁹Fe), lead (²⁰³Pb, ²¹⁰Pb, ²¹²Pb), lutetium (¹⁷⁷Lu), magnesium (⁵²Mg), manganese (⁵⁴Mn), mercury (¹⁹⁷Hg, ²⁰³Hg), molybdenum (⁹⁹Mo), neodynium (¹⁴⁷Nd), neptunium (²³⁷Np), nickel (⁵⁷Ni, ⁶³Ni), niobium (⁹⁵Nb), osmium (¹⁸⁵Os, ¹⁹¹Os), palladium (¹⁰³Pd, ¹⁰⁹Pd), phosphorus (³²P, ³³P), platinum (¹⁹⁵Pt, ¹⁹⁷Pt), plutonium (²³⁹Pu), potassium (⁴⁰K), praseodynium (¹⁴²Pr, ¹⁴³Pr), promethium (¹⁴⁷Pm), protactinium (²³³Pa), radium (²²³Ra, ²²⁶Ra), rhenium (¹⁸⁶Re, ¹⁸⁸Re), rhodium (¹⁰⁵Rh), rubidium (⁸¹Rb, ⁸⁶Rb), ruthenium (⁹⁵Ru, ⁹⁷Ru, ¹⁰³Ru, ¹⁰⁵Ru, ¹⁰⁶Ru), samarium (¹⁵³Sm), scandium (⁴⁴Sc, ⁴⁶Sc, ⁴⁷Sc), selenium (⁷²Se, ⁷³Se, ⁷⁵Se), silver (¹⁰⁰Ag, ¹¹¹Ag), sodium (²²Na), strontium (⁸⁵Sr, ⁸⁹Sr, ⁹⁰Sr), sulfur (³⁵S), tantalum (¹⁷⁹Ta, ¹⁸²Ta), technetium (⁹⁹Tc), tellurium (¹²¹Te, ¹²²Te, ¹²⁵Te, ¹³²Te), terbium (¹⁶¹Tb), thalium (¹⁷⁰Tl, ²⁰¹Tl, ²⁰⁴Tl), thorium (²²⁸Th, ²³⁰Th, ²³²Th), thulium (¹⁶⁵Tm, ^{167 Tm,} ¹⁶⁸Tm, ¹⁷⁰Tm), tin (¹¹³Sn), titanium (⁴⁴Ti), tungsten (¹⁸⁵W), uranium(²³³U, ²³⁵U, ²³⁸U), vanadium (⁴⁸V, ⁴⁹V), ytterbium (¹⁶⁹Yb), yttrium (⁸⁸Y, ⁹⁰Y, ⁹¹Y), zinc (⁶²Zn, ⁶⁵Zn) and zirconium (⁹⁵Zr).

12. The method of claim 1, wherein said biological agents are selected from the group consisting of anthrax and its toxins, botulinum and its toxins, aflatoxin, sterigmatocystin, deoxynivalenol, fumonisin B1, Clostridium dificile and its toxins, plague (Yersinia pestis) and its toxins, hemorrhagic fevers, Staphylococcus aureus, Streptococcus, ricin, modeccin, diphtheria, and Pseudomonas, and cholera and its toxins.

13. The method of claim 1, wherein said chemical agents are selected from the group consisting of phosphoramide mustard, melphalan, chlorambucil, quinacrine mustard, nitrogen mustard, cyclophosphamide, 4-hydroxycyclophosphamide, and cyanide.

14. The method of claim 1, wherein said caspase inhibitor is administered after exposure to radionuclides, biological agents, or chemical agents in said animal.

15. The method of claim 1, wherein said caspase inhibitor is administered during exposure to radionuclides, biological agents, or chemical agents in said animal.

16. The method of claim 1, wherein said caspase inhibitor is administered prior to exposure to radionuclides, biological agents, or chemical agents in said animal.

17. The method of claim 1, wherein said caspase inhibitor has the formula: 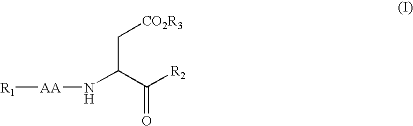
or a pharmaceutically acceptable salt thereof;
wherein R₁ is an N-terminal protecting group;
AA is a residue of any natural or non-natural α-amino acid, β-amino acid, derivatives of an α-amino acid or β-amino acid;
R₂ is H or CH₂R₄ where R₄ is an electronegative leaving group; and
R₃ is alkyl or H.

18. The method of claim 17, wherein said caspase inhibitor is Boc-Ala-Asp-CH₂F, Boc-Val-Asp-CH₂F, Boc-Leu-Asp-CH₂F, Ac-Val-Asp-CH₂F, Ac-Ile-Asp-CH₂F, Ac-Met-Asp-CH₂F, Cbz-Val-Asp-CH₂F, Cbz-β-Ala-Asp-CH₂F, Cbz-Leu-Asp-CH₂F, Cbz-Ile-Asp-CH₂F, Boc-Ala-Asp(OMe)-CH₂F, Boc-Val-Asp(OMe)-CH₂F, Boc-Leu-Asp(OMe)-CH₂F, Ac-Val-Asp(OMe)-CH₂F, Ac-Ile-Asp(OMe)-CH₂F, Ac-Met-Asp(OMe)-CH₂F, Cbz-Val-Asp(OMe)-CH₂F, Cbz-β-Ala-Asp(OMe)-CH₂F, Cbz-Leu-Asp(OMe)-CH₂F or Cbz-Ile-Asp(OMe)-CH₂F.

19. The method of claim 1, wherein said caspase inhibitor has the formula II: 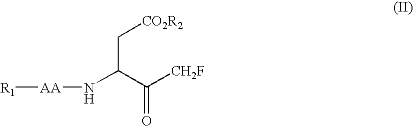
or a pharmaceutically acceptable salt thereof;
wherein R₁ is an N-terminal protecting group;
AA is a residue of a non-natural α-amino acid or β-amino acid; and
R₂ is an optionally substituted alkyl or H.

20. The method of claim 19, wherein said caspase inhibitor is Boc-Phg-Asp-fmk, Boc-(2-F-Phg)-Asp-fmk, Boc-(F₃-Val)-Asp-fmk, Boc-(3-F-Val)-Asp-fmk, Ac-Phg-Asp-fmk, Ac-(2-F-Phg)-Asp-fmk, Ac-(F₃-Val)-Asp-fmk, Ac-(3-F-Val)-Asp-fmk, Z-Phg-Asp-fmk, Z-(2-F-Phg)-Asp-fmk, Z-(F₃-Val)-Asp-fmk, Z-Chg-Asp-fmk, Z-(2-Fug)-Asp-fmk, Z-(4-F-Phg)-Asp-fmk, Z-(4-Cl-Phg)-Asp-fmk, Z-(3-Thg)-Asp-fmk, Z-(2-Fua)-Asp-fmk, Z-(2-Tha)-Asp-fmk, Z-(3-Fua)-Asp-fmk, Z-(3-Tha)-Asp-fmk, Z-(3-Cl-Ala)-Asp-fmk, Z-(3-F-Ala)-Asp-fmk, Z-(F₃-Ala)-Asp-fmk, Z-(3-F-3-Me-Ala)-Asp-fmk, Z-(3-Cl-3-F-Ala)-Asp-fmk, Z-(2-Me-Val)-Asp-fmk, Z-(2-Me-Ala)-Asp-fmk, Z-(2-i-Pr-β-Ala)-Asp-fmk, Z-(3-Ph-β-Ala)-Asp-fmk, Z-(3-CN-Ala)-Asp-fmk, Z-(1-Nal)-Asp-fmk, Z-Cha-Asp-fmk, Z-(3-CF₃-Ala)-Asp-fmk, Z-(4-CF₃-Phg)-Asp-fmk, Z-(3-Me₂N-Ala)-Asp-fmk, Z-(2-Abu)-Asp-fmk, Z-Tle-Asp-fmk, Z-Cpg-Asp-fmk, Z-Cbg-Asp-fmk, Z-Thz-Asp-fmk, Z-(3-F-Val)-Asp-fmk, or Z-(2-Thg)-Asp-fmk.

21. The method of claim 1, wherein said caspase inhibitor has the formula of one of III, IV and V: 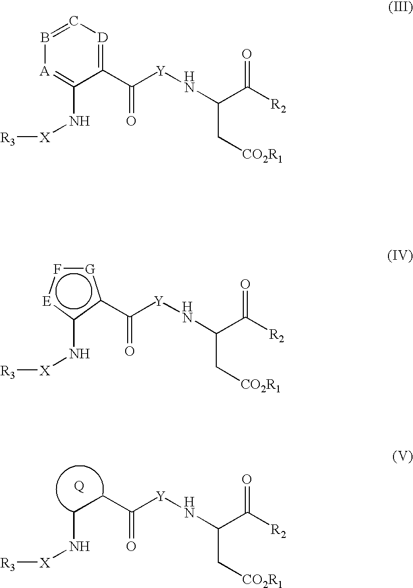
or a pharmaceutically acceptable salt thereof;
wherein R₁ is an optionally substituted alkyl or hydrogen,
R₃ is an N-protecting group;
R₂ is hydrogen or optionally substituted alkyl;
A is CR₆ or nitrogen;
B is CR₇ or nitrogen;
C is CR₈ or nitrogen;
D is CR₉ or nitrogen;
provided that not more than two of A, B, C or D is nitrogen; and
R₆-R₉ independently are hydrogen, halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl; C₁-C₆ hydroxyalkyl, nitro, amino, cyano, C₁-C₆ acylamino, hydroxy, C₁-C₆ acyloxy, C₁-C₆ alkoxy, alkylthio, or carboxy; or
one of R₆ and R₇, or R₇ and R₈, or R₈ and R₉ are taken together with the carbon atoms to which they are attached to form a carbocycle or heterocycle;
E is CR₁₄, nitrogen, oxygen or sulfur;
F is CR₁₅, nitrogen, oxygen or sulfur;
G is C₁₆, nitrogen, oxygen or sulfur;
provided that only one of E, F, G is nitrogen, oxygen or sulfur, where R₁₄-R₁₆ are independently hydrogen, halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl; C₁-C₆ hydroxyalkyl, nitro, amino, cyano, C₁-C₆ acylamino, hydroxy, C₁-C₆ acyloxy, C₁-C₆ alkoxy, alkylthio, or carboxy; or
one of R₁₄ and R₁₅, or R₁₅ and R₁₆, are taken together with the carbon atoms to which they are attached to form a carbocycle or heterocycle;
Q represents an optionally substituted saturated or partially saturated carbocycle or heterocycle;
X is a peptide of 1-4 amino acids or a bond; and
Y is a peptide of 1-4 amino acids or a bond.

22. The method of claim 21, wherein said caspase inhibitor is 2-(Z-amino)benzoyl-Asp-fmk, 2-(Z-amino)-3-methylbenzoyl-Asp-fmk, 2-(Z-amino)-3,5-dimethylbenzoyl-Asp-fmk, 2-(Z-amino)-4-chlorobenzoyl-Asp-fmk, 2-(Z-amino)-5-chlorobenzoyl-Asp-fmk, 2-(Z-amino)-5-fluorobenzoyl-Asp-fmk, 2-(Z-amino)-6-fluorobenzoyl-Asp-fmk, cis-2-(Z-amino)cyclohexanecarboxyl-Asp-fmk, 2-(Z-amino)-5-methylbenzoyl-Asp-fmk, 2-(Z-amino)-6-methylbenzoyl-Asp-fmk, 2-(Z-amino)-6-chlorobenzoyl-Asp-fmk, 2-(Z-amino)-3-methoxybenzoyl-Asp-fmk, 2-(Z-amino)thiophene-2-carboxyl-Asp-fmk, 2-(methoxycarbonylamino)thiophene-2-carboxyl -Asp-fmk, cis-2-(Z-amino)cyclopentanecarboxyl-Asp-fmk, trans-2-(Z-amino)cyclopentanecarboxyl-Asp-fmk, 2-(Z-amino)benzoyl-Asp-DCB-methylketone, methoxycarbonyl-Val-(2-aminobenzoyl)-Asp-fmk, Z-Glu-(2-aminobenzoyl)-Asp-fmk or Z-Val-(2-aminobenzoyl)-Asp-fmk.

23. The method of claim 1, wherein said caspase inhibitor has the formula VI: 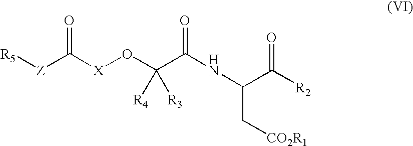
or a pharmaceutically acceptable salt thereof, wherein
R₁ is an optionally substituted alkyl or hydrogen;
R₂ is hydrogen or optionally substituted alkyl;
R₃ and R₄ independently are hydrogen, optionally substituted aryl, optionally substituted heterocyclic, optionally substituted carbocyclic, optionally substituted heteroaryl, optionally substituted alkyl, optionally substituted alkenyl, or optionally substituted alkynyl;
R₅ is an optionally substituted alkyl, optionally substituted carbocyclic, optionally substituted heterocyclic, optionally substituted aryl or optionally substituted heteroaryl;
Z is O, S, NR₈, or (CR₉R₁₀)ₙ, where R₈, R₉ and R₁₀ independently are hydrogen, alkyl or cycloalkyl, and n is 0, 1, 2, or 3; and
X is a peptide of 1-2 amino acids or a bond.

24. The method of claim 23, wherein said caspase inhibitor is 1-(Carbonyl-Asp-CH₂F)ethyl N-phenylcarbamate, 1-(Carbonyl-Asp-CH₂F)ethyl N-benzylcarbamate, 2-Methyl-1-(carbonyl-Asp-CH₂F)propyl N-phenylcarbamate, 2-Methyl-1-(carbonyl-Asp-CH₂F)propyl N-benzylcarbamate, 2-Methyl-1-(carbonyl-Asp-CH₂F)propyl N-(2,6-dichlorophenyl)carbamate, 2-Methyl-1-(carbonyl-Asp-CH₂F)propyl N-(2,5-dichlorophenyl)-carbamate, 2-Methyl-1-(carbonyl-Asp-CH₂F)propyl N-(2,4-dichlorophenyl)-carbamate, 2-Methyl-1-(carbonyl-Asp-CH₂DCB)propyl N-phenylcarbamate, 2-Methyl-1-(carbonyl-Asp-CH₂DCB)propyl N-(2,6-dichlorophenyl)-carbamate, 2-Methyl-1-(carbonyl-Asp-CH₂PTP)propyl N-phenylcarbamate, 2-Methyl-1-(carbonyl-Asp-CH₂PTP)propyl N-(2,6-dichlorophenyl)-carbamate, 2-Methyl-1-(carbonyl-Asp-CH₂DPP)propyl N-phenylcarbamate, 2-Methyl-1-(carbonyl-Asp-CH₂DPP)propyIN-(2,6-dichlorophenyl)-carbamate, 2-Methyl-1-(carbonyl-Asp-CH₂F)propyl N-(2-methyl-1-methoxycarbonyl-propyl)carbamate, 2-Methyl-1-(carbonyl-Asp-CH₂F)propyl N-(3-fluorophenyl)carbamate, 2-Methyl-1-(carbonyl-Asp-CH₂F)propyl N-(4-fluorophenyl)carbamate, 2-Methyl-1-(carbonyl-Asp-CH₂F)propyl N-(3,4-difluorophenyl)carbamate, 2-Methyl-1-(carbonyl-Asp-CH₂F)propyl N-(4-phenoxyphenyl)carbamate, 1-(Carbonyl-Asp-CH₂F)propyl N-phenylcarbamate, 1-(Carbonyl-Asp-CH₂F)butyl N-phenylcarbamate, 1-(Carbonyl-Asp-CH₂F)-2-propenyl N-phenylcarbamate, 2-(4-Imidazolyl)-1-(carbonyl-Asp-CH₂F)ethyl N-phenylcarbamate, 2-Phenyl-1-(carbonyl-Asp-CH₂F)ethyl N-phenylcarbamate, 2-Methyl-1-(carbonyl-Asp-CH₂F)butyl N-phenylcarbamate, 3-Methyl-1-(carbonyl-Asp-CH₂F)butyl N-phenylcarbamate, 1-Phenyl-1-(carbonyl-Asp-CH₂F)methyl N-phenylcarbamate, 1-(2-Chlorophenyl)-1-(carbonyl-Asp-CH₂F)methyl N-phenylcarbamate, 1-(4-Chlorophenyl)-1-(carbonyl-Asp-CH₂F)methyl N-phenylcarbamate, 1-Cyclohexyl-1-(carbonyl-Asp-CH₂F)methyl N-phenylcarbamate, 2-Chloro-1-(carbonyl-Asp-CH₂F)ethyl N-phenylcarbamate, 2,2,2-Trifluoro-1-(carbonyl-Asp-CH₂F)ethyl N-phenylcarbamate or Z-Valine 2-methyl-1-(carbonyl-Asp-CH₂F)propyl ester.

25. The method of claim 1, wherein said caspase inhibitor has the formula VII: 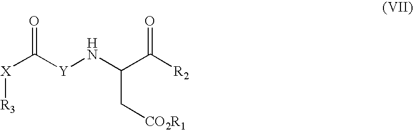
or a pharmaceutically acceptable salt thereof;
wherein R₁ is an optionally substituted alkyl or hydrogen;
R₂ is hydrogen or optionally substituted alkyl;
R₃ is an alkyl, saturated carbocyclic, partially saturated carbocyclic, aryl, saturated heterocyclic, partially saturated heterocyclic or heteroaryl group, wherein said group is optionally substituted;
X is O, S, NR₄, or (CR₄R₅)ₙ, where R₄ and R₅ are, at each occurrence, independently selected from the group consisting of hydrogen, alkyl and cycloalkyl, and n is 0, 1, 2, or 3; or
X is NR₄, and R₃ and R₄ are taken together with the nitrogen atom to which they are attached to form a saturated heterocyclic, partially saturated heterocyclic or heteroaryl group, wherein said group is optionally substituted; or
X is CR₄R₅, and R₃ and R₄ are taken together with the carbon atom to which they are attached to form a saturated carbocyclic, partially saturated carbocyclic, aryl, saturated heterocyclic, partially saturated heterocyclic or oxygen-containing heteroaryl group, wherein said group is optionally substituted; and
Y is a residue of a natural or non-natural amino acid;
provided that when X is O, then R₃ is not unsubstituted benzyl or t-butyl; and when X is CH₂, then R₃ is not hydrogen.

26. The method of claim 25, wherein said caspase inhibitor is 2-Chlorobenzyloxycarbonyl-Val-Asp-fmk, 3-Chlorobenzyloxycarbonyl-Val-Asp-fmk, 4-Chlorobenzyloxycarbonyl-Val-Asp-fmk, Phenethoxycarbonyl-Val-Asp-fmk, Cyclohexylmethoxycarbonyl-Val-Asp-fmk, Methoxycarbonyl-Val-Asp-fmk, Ethoxycarbonyl-Val-Asp-fmk, Isopropyloxycarbonyl-Val-Asp-fmk, 2-Chlorobenzyloxycarbonyl-Ile-Asp-fmk, 3-Chlorobenzyloxycarbonyl-Ile-Asp-fmk, 4-Chlorobenzyloxycarbonyl-Ile-Asp-fmk, Phenylacetyl-Val-Asp-fmk, 4-Nitrobenzyloxycarbonyl-Val-Asp-fmk, 2,5-Dimethylbenzyloxycarbonyl-Val-Asp-fmk, 3,4-Dichlorobenzyloxycarbonyl-Val-Asp-fmk, 3,5-Dichlorobenzyloxycarbonyl-Val-Asp-fmk, 2,5-Dichlorobenzyloxycarbonyl-Val-Asp-fmk, 2,6-Dichlorobenzyloxycarbonyl-Val-Asp-fmk, 2,4-Dichlorobenzyloxycarbonyl-Val-Asp-fmk, 2,4-Dimethylbenzyloxycarbonyl-Val-Asp-fmk, 4-Ethylbenzyloxycarbonyl-Val-Asp-fmk, 4-Bromobenzyloxycarbonyl-Val-Asp-fmk, 4-Fluorobenzyloxycarbonyl-Val-Asp-fmk, Cyclopentylmethoxycarbonyl-Val-Asp-fmk, 4-Trifluoromethylbenzyloxycarbonyl-Val-Asp-fmk, 3-Phenylpropionyl-Val-Asp-fmk, Benzylaminocarbonyl-Val-Asp-fmk, 3-Phenylpropyloxycarbonyl-Val-Asp-fmk, 2,4-Difluorobenzyloxycarbonyl-Val-Asp-fmk, 3,4-Difluorobenzyloxycarbonyl-Val-Asp-fmk, 4-Morpholinecarbonyl-Val-Asp-fmk, 4-Pyridylmethoxycarbonyl-Val-Asp-fmk, 2-Pyridylmethoxycarbonyl-Val-Asp-fmk, 2,6-Dichlorobenzyloxycarbonyl-Val-Asp-DCB-methylketone, Isobutoxycarbonyl-Val-Asp-fmk, Propionyl-Val-Asp-fmk, Benzyl-glutaryl-Val-Asp-fmk,Glutaryl-Val-Asp-fmk, 3-(2-Phenyloxyphenyl)propionyl-Val-Asp-fmk, 3-(5-Bromo-2-hydroxyphenyl)propionyl-Val-Asp-fmk, 3-Fluorobenzyloxycarbonyl-Val-Asp-fmk, 2-Fluorobenzyloxycarbonyl-Val-Asp-fmk, 3-Methylbenzyloxycarbonyl-Val-Asp-fmk, 2-Chloro-4-fluorobenzyloxycarbonyl-Val-Asp-fmk, 2-Naphthylmethoxycarbonyl-Val-Asp-fmk, p-Toluenesulfonyl-Val-Asp-fmk or p-Toluenesulfonyl-Phe-Asp-fmk.
